(19) United States
(12) Patent Application Publication
(10) Pub. No.: US 2024/0350771 A1
(43) Pub. Date: Oct. 24, 2024

(54) MEDICAL SYSTEM

(71) Applicant: CANON KABUSHIKI KAISHA, Tokyo (JP)

(72) Inventors: RYO IWASAWA, Kanagawa (JP); ISAO MATSUOKA, Kanagawa (JP); KOSUKE FUJIMOTO, Kanagawa (JP); YASUMICHI ARIMITSU, Tochigi (JP); DAISUKE KANEKO, Tokyo (JP)

(21) Appl. No.: 18/758,415

(22) Filed: Jun. 28, 2024

Related U.S. Application Data
(63) Continuation of application No. PCT/JP2022/047522, filed on Dec. 23, 2022.

(30) Foreign Application Priority Data
Jan. 14, 2022 (JP) 2022-004641

(51) Int. Cl. A61M 25/01

(52) U.S. Cl. CPC A61M 25/0113; A61M 2205/332; A61M 2205/3327

(57) ABSTRACT

A medical system includes a driving unit including a drive source and a first member, a bendable unit that includes a bending portion, a linear body, and a second member and that is attachable to and detachable from the driving unit, and a control portion. In a state in which the bendable unit is attached to the driving unit, the first member and the second member engages with each other such that the first member and the second member integrally move in the longitudinal direction. The control portion is configured to, in a case where the bendable unit is attached to the driving unit, move the first member toward a position in the longitudinal direction where the first member is capable of engaging with the second member, by the drive source and in accordance with a position of the second member in the longitudinal direction.

### CROSS-REFERENCE TO RELATED APPLICATIONS

This application is a Continuation of International Patent Application No. PCT/JP2022/047522, filed Dec. 23, 2022, which claims the benefit of Japanese Patent Application No. 2022-004641, filed Jan. 14, 2022, both of which are hereby incorporated by reference herein in their entirety.

### BACKGROUND OF THE INVENTION

### Field of the Invention

The present invention relates to a medical system including a bendable unit that is capable of deforming by bending.

### Description of the Related Art

For a medical apparatus including a bendable unit that is capable of deforming by bending such as a catheter unit, a configuration in which a bendable unit is attachable to and detachable from a base unit (actuator body portion) including a drive source is known. Patent Literature 1 discloses a configuration in which a catheter unit can be attached and detached by rotating the catheter unit with respect to the actuator body portion. According to this document, a rod coupled to a driving wire of a catheter is provided on the catheter unit side, the rod is fitted in a groove provided on a driving stage on the actuator body portion side, and thus the bending of the catheter can be controlled by the driving force of a drive source.

### CITATION LIST

### Patent Literature

Patent Literature 1: US Patent Application Publication No. 2021/0121051

In the configuration described in the document described above, the position of the rod varies at the time before attaching the catheter unit to the actuator body portion, due to a reason such as the manufacture tolerance of the catheter unit. If the rod interferes with a member on the actuator body portion side at an unexpected position at the time of attachment of the catheter unit due to the positional deviation of the rod, there is a possibility that the rod fails to fit in the groove on the driving stage or part of the apparatus is damaged.

In the document described above, an end portion of the groove of the driving stage is chamfered so as to absorb the positional deviation of the rod in the longitudinal direction to facilitate the fitting. However, in the case where the position of the rod is deviated greatly, there is a possibility that a range where the deviation can be addressed by the chamfered shape is exceeded.

### SUMMARY OF THE INVENTION

The present invention provides a medical system with which attachment of a bendable unit can be performed more reliably.

An aspect of the present invention is a medical system including a driving unit including a drive source and a first member connected to the drive source, a bendable unit that includes a bending portion capable of bending, a linear body configured to bend the bending portion, and a second member connected to the linear body and that is attachable to and detachable from the driving unit, and a control portion configured to control the drive source, wherein the second member includes a first abutted portion and a second abutted portion, wherein the first member includes a first abutting portion configured to abut the first abutted portion and press the second member toward one side in a longitudinal direction of the linear body, and a second abutting portion configured to abut the second abutted portion and press the second member toward an other side in the longitudinal direction, wherein, in a state in which the bendable unit is attached to the driving unit, the first member and the second member are configured to engage with each other such that the first abutting portion and the second abutting portion respectively abut the first abutted portion and the second abutted portion so that the first member and the second member integrally move in the longitudinal direction, and wherein the control portion is configured to, in a case where the bendable unit is attached to the driving unit, move the first member toward a position in the longitudinal direction where the first member is capable of engaging with the second member, by using the drive source and in accordance with a position of the second member in the longitudinal direction.

Further features of the present invention will become apparent from the following description of exemplary embodiments with reference to the attached drawings. To be noted, in the attached drawings, the same or substantially the same elements are denoted by the same reference numerals.

### BRIEF DESCRIPTION OF THE DRAWINGS

FIG. 1 is an overall view of a medical system.

FIG. 2 is a perspective view of a medical apparatus and a supporting base.

FIG. 3A an explanatory diagram of a catheter.

FIG. 3B is an explanatory diagram of the catheter.

FIG. 4A is an explanatory diagram of a catheter unit.

FIG. 4B is an explanatory diagram of the catheter unit.

FIG. 5A is an explanatory diagram of a base unit and a wire driving portion.

FIG. 5B is an explanatory diagram of the base unit and the wire driving portion.

FIG. 5C is an explanatory diagram of the base unit and the wire driving portion.

FIG. 6A is an explanatory diagram of the wire driving portion, a coupling device, and a bending driving portion.

FIG. 6B is an explanatory diagram of the wire driving portion, the coupling device, and the bending driving portion.

FIG. 6C is an explanatory diagram of the wire driving portion, the coupling device, and the bending driving portion.

FIG. 7A is an explanatory diagram of attachment of the catheter unit.

FIG. 7B is an explanatory diagram of attachment of the catheter unit.

FIG. 8A is a diagram for describing coupling between the catheter unit and the base unit.

FIG. 8B is a diagram for describing coupling between the catheter unit and the base unit.

FIG. 9 is an exploded view for describing coupling between the catheter unit and the base unit.

FIG. 10A is an explanatory diagram of fixation of a driving wire via a coupling portion.

FIG. 10B is an explanatory diagram of fixation of the driving wire via the coupling portion.

FIG. 11 is an explanatory diagram of fixation of the driving wire via the coupling portion.

FIG. 12 is an explanatory diagram of fixation of the driving wire via the coupling portion.

FIG. 13 is an explanatory diagram of fixation of the driving wire via the coupling portion.

FIG. 14A is an explanatory diagram of fixation of the driving wire via the coupling portion.

FIG. 14B is an explanatory diagram of fixation of the driving wire via the coupling portion.

FIG. 15 is an explanatory diagram of fixation of the driving wire via the coupling portion.

FIG. 16A is an explanatory diagram of the catheter unit and the base unit.

FIG. 16B is an explanatory diagram of the catheter unit and the base unit.

FIG. 16C is an explanatory diagram of the catheter unit and the base unit.

FIG. 17A is a diagram for describing an operation of an operation portion.

FIG. 17B is a diagram for describing the operation of the operation portion.

FIG. 17C is a diagram for describing the operation of the operation portion.

FIG. 18A is a section view for describing the operation of the operation portion.

FIG. 18B is a section view for describing the operation of the operation portion.

FIG. 18C is a section view for describing the operation of the operation portion.

FIG. 19A is an explanatory diagram of a positional deviation correction configuration according to Example 1.

FIG. 19B is an explanatory diagram of the positional deviation correction configuration according to Example 1.

FIG. 20 is an explanatory diagram of the positional deviation correction configuration according to Example 1.

FIG. 21 is an explanatory diagram of a locked state of the coupling portion according to Example 1.

FIG. 22 is an explanatory diagram of a positional deviation correction configuration according to a modification example.

FIG. 23 is an explanatory diagram of a positional deviation correction configuration according to Example 2.

FIG. 24A is an explanatory diagram of the positional deviation correction configuration according to Example 2.

FIG. 24B is an explanatory diagram of the positional deviation correction configuration according to Example 2.

FIG. 25 is an explanatory diagram of a positional deviation correction configuration according to Example 3.

FIG. 26A is an explanatory diagram of the positional deviation correction configuration according to Example 3.

FIG. 26B is an explanatory diagram of the positional deviation correction configuration according to Example 3.

### DESCRIPTION OF THE EMBODIMENTS

Embodiments of the present disclosure will be described below with reference to drawings. To be noted, the dimensions, materials, shapes, and arrangements of constituent parts described in the embodiments should be appropriately modified in accordance with the configuration and various conditions of the apparatus to which the present technique is applied.

### Example 1

### <Medical System and Medical Apparatus>

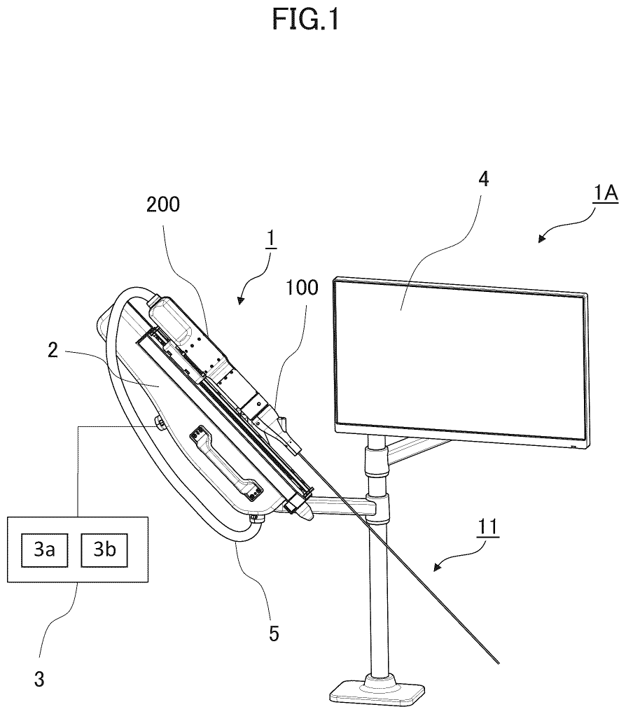

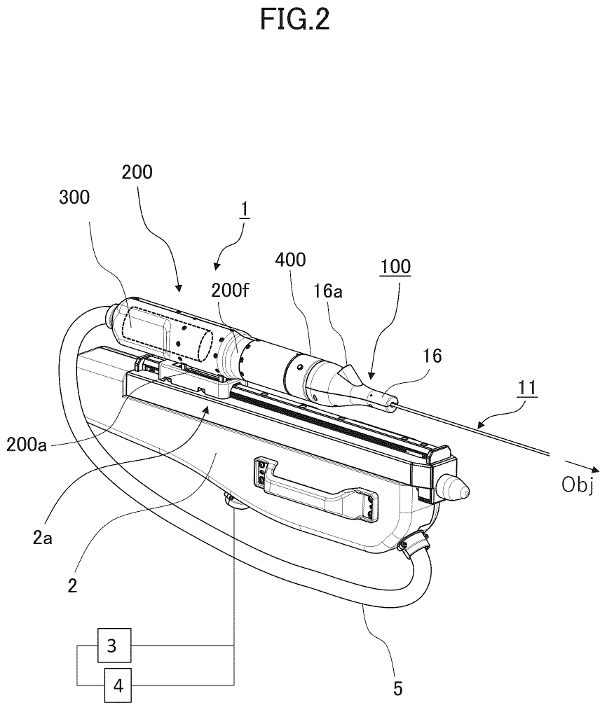

A medical system 1A and a medical apparatus 1 will be described with reference to FIGS. 1 and 2. FIG. 1 is an overall view of the medical system 1A. FIG. 2 is a perspective view of the medical apparatus 1 and a supporting base 2.

The medical system 1A includes the medical apparatus 1, the supporting base 2 to which the medical apparatus 1 is attached, and a control portion (control apparatus, controller) 3 that controls the medical apparatus 1. In the present example, the medical system 1A includes a monitor 4 serving as a display apparatus.

The medical apparatus 1 includes a catheter unit (bendable unit) 100 including a catheter 11 serving as a bendable body, and a base unit (driving unit, attached unit) 200. The catheter unit 100 is configured to be attachable to and detachable from the base unit 200.

In the present example, the user of the medical system 1A and the medical apparatus 1 is capable of performing a work such as observing the inside of an object Obj, collecting various specimens from the inside of the object Obj, and treating the inside of the object Obj, by inserting the catheter 11 in the object Obj. As one embodiment, the user can insert the catheter 11 in a patient serving as the object Obj. Specifically, by insertion in a bronchial tube through the mouth or nose of the patient, a work such as observing, collecting, or cutting a lung tissue can be performed.

The catheter 11 can be used as a guide (sheath) for guiding a medical tool for performing the work described above. As examples of the medical tool (tool), endoscopes, forceps, and abrasion devices can be mentioned. In addition, the bendable body itself may have a function as the medical tools described above, and in this case, the bendable body is not limited to a tubular shape and may have, for example, a columnar shape.

In the present example, the control portion 3 includes a calculation apparatus 3a, and an input apparatus 3b. The input apparatus 3b receives commands and input for operating the catheter 11. The calculation apparatus 3a includes a storage that stores programs and various data for controlling the catheter, a random access memory, and a central processing apparatus for executing the program. In addition, the control portion 3 may include an output portion that outputs a signal for displaying an image on the monitor 4.

As illustrated in FIG. 2, in the present example, the medical apparatus 1 is electrically connected to the control portion 3 via the supporting base 2 and a cable 5 interconnecting a base unit 200 of the medical apparatus 1 and the supporting base 2. To be noted, the medical apparatus 1 and the control portion 3 may be directly interconnected by a cable. The medical apparatus 1 and the control portion 3 may be wirelessly connected to each other.

The medical apparatus 1 is detachably attached to the supporting base 2 via the base unit 200. More specifically, regarding the medical apparatus 1, an attachment portion (connecting portion) 200a of the base unit 200 is detachably attached to a moving stage (receiving portion) 2a of the supporting base 2. Even in a state in which the attachment portion 200a of the medical apparatus 1 is detached from the moving stage 2a, the connection between the medical apparatus 1 and the control portion 3 is maintained such that the medical apparatus 1 can be controlled by the control portion 3. In the present example, the medical apparatus 1 and the supporting base 2 are interconnected via the cable 5 even in a state in which the attachment portion 200a of the medical apparatus 1 is detached from the moving stage 2a.

The user can insert the catheter 11 in the object Obj by manually moving the medical apparatus 1 in a state in which the medical apparatus 1 is detached from the supporting base 2 (state in which the medical apparatus 1 is detached from the moving stage 2a).

The user can use the medical apparatus 1 in a state in which the catheter 11 is inserted in the object Obj and the medical apparatus 1 is attached to the supporting base 2. Specifically, the medical apparatus 1 moves as a result of the moving stage 2a moving in a state in which the medical apparatus 1 is attached to the moving stage 2a. Then, the operation of moving the catheter 11 in a direction to insert the catheter 11 in the object Obj and the operation of moving the catheter 11 in a direction to pull out the catheter 11 from the object Obj are performed. The movement of the moving stage 2a is controlled by the control portion 3.

The medical apparatus 1 includes a wire driving portion (linear member driving portion, line driving portion, body driving portion) 300 for driving the catheter 11. In the present example, the medical apparatus 1 is a robot catheter apparatus that drives the catheter 11 by the wire driving portion 300 controlled by the control portion 3.

The control portion 3 is capable of performing an operation of bending the catheter 11 by controlling the wire driving portion 300. In the present example, the wire driving portion 300 is included in the base unit 200. More specifically, the base unit 200 includes a base casing 200f accommodating the wire driving portion 300. That is, the base unit 200 includes the wire driving portion 300. The wire driving portion 300 and the base unit 200 can be collectively referred to as a catheter driving apparatus (base apparatus, main body).

In an extending direction of the catheter 11, an end portion where the distal end of the catheter 11 inserted in the object Obj will be referred to as a far end. The opposite side of the far end in the extending direction of the catheter 11 will be referred to as a near end.

The catheter unit 100 includes a near-end cover 16 that covers the near-end side of the catheter 11. The near-end cover 16 has a tool hole 16a. A medical tool can be inserted in the catheter 11 through the tool hole 16a.

As described above, in the present example, the catheter 11 has a function as a guide apparatus for guiding the medical tool to a desired position in the object Obj.

For example, the catheter 11 is inserted to a target position in the object Obj in a state in which an endoscope is inserted in the catheter 11. At this time, at least one of a manual operation by the user, movement of the moving stage 2a, and driving of the catheter 11 by the wire driving portion 300 is used. After the catheter 11 has reached the target position, the endoscope is pulled out of the catheter 11 through the tool hole 16a. Then, the medical tool is inserted through the tool hole 16a, and a work such as collection of various specimens from the inside of the object Obj or treatment of the inside of the object Obj is performed.

As will be described later, the catheter unit 100 is detachably attached to the catheter driving apparatus (base apparatus, main body), more specifically to the base unit 200. After the medical apparatus 1 is used, the user detaches the catheter unit 100 from the base unit 200, attaches a new catheter unit 100 to the base unit 200, and thus it becomes possible to use the medical apparatus 1 again. That is, the catheter unit 100 can be used as a disposable unit. Here, disposable unit that the catheter unit 100 used in one time of treatment is disposed after being used. As a result of this, reuse of the catheter unit 100 is prevented, and thus the medical apparatus 1 can be always maintained in a clean state.

As illustrated in FIG. 2, the medical apparatus 1 includes an operation portion 400. In the present example, the operation portion 400 is provided in the catheter unit 100. The operation portion 400 is operated by the user when fixing the catheter unit 100 to the base unit 200 or detaching the catheter unit 100 from the base unit 200.

By connecting the endoscope inserted in the catheter 11 to the monitor 4, an image captured by the endoscope can be displayed on the monitor 4. In addition, by connecting the monitor 4 to the control portion 3, the state of the medical apparatus 1 and information related to the control of the medical apparatus 1 can be displayed on the monitor 4. For example, the position of the catheter 11 in the object Obj and information related to navigation of the catheter 11 in the object Obj can be displayed on the monitor 4. The monitor 4, the control portion 3, and the endoscope may be connected to each other in a wired manner or in a wireless manner. In addition, the monitor 4 and the control portion 3 may be connected to each other via the supporting base 2.

### <Catheter>

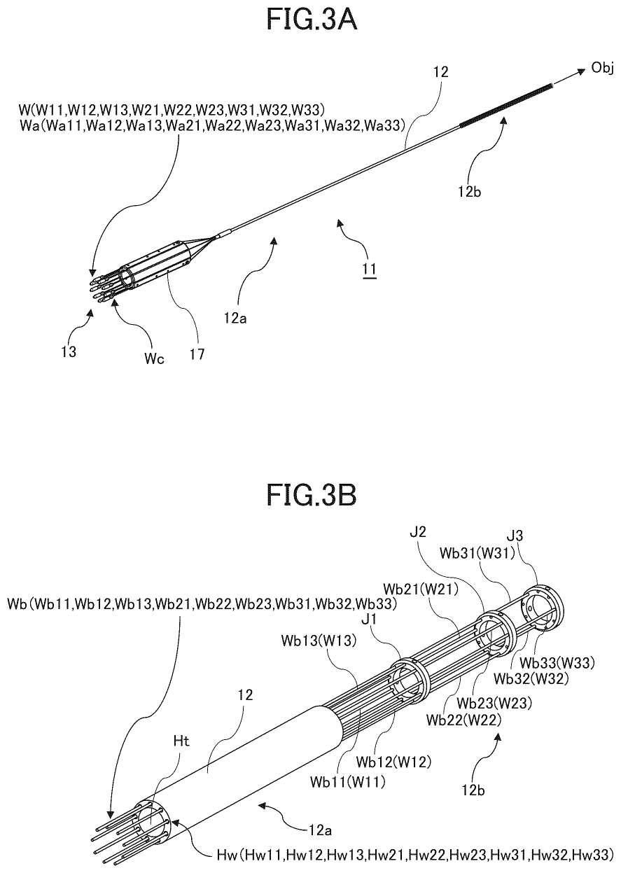

The catheter 11 serving as a bendable body will be described with reference to FIGS. 3A and 3B. FIGS. 3A and 3B are explanatory diagrams of the catheter 11. FIG. 3A is a diagram for describing the entirety of the catheter 11. FIG. 3B is an enlarged view of the catheter 11.

The catheter 11 includes a bending portion (bending body, catheter body) 12, and a bending driving portion (catheter driving portion) 13 configured to bend the bending portion 12. The bending driving portion 13 is configured to bend the bending portion 12 by receiving a driving force of the wire driving portion 300 via a coupling device 21 that will be described later.

The catheter 11 extends in the insertion direction of the catheter 11 with respect to the object Obj. The extending direction (longitudinal direction) of the catheter 11 is the same as the extending direction (longitudinal direction) of the bending portion 12 and the extending direction (longitudinal direction) of first to ninth driving wires (W11 to W33) that will be described later.

The bending driving portion 13 includes a plurality of driving wires (driving lines, linear members, linear actuators) connected to the bending portion 12. Specifically, the bending driving portion 13 includes a first driving wire W11, a second driving wire W12, a third driving wire W13, a fourth driving wire W21, a fifth driving wire W22, a sixth driving wire W23, a seventh driving wire W31, an eighth driving wire W32, and a ninth driving wire W33.

The first to ninth driving wires (W11 to W33) each include a held portion (held shaft, rod) Wa. Specifically, the first driving wire W11 includes a first held portion Wa11. The second driving wire W12 includes a second held portion Wa12. The third driving wire W13 includes a third held portion Wa13. The fourth driving wire W21 includes a fourth held portion Wa21. The fifth driving wire W22 includes a fifth held portion Wa22. The sixth driving wire W23 includes a sixth held portion Wa23. The seventh driving wire W31 includes a seventh held portion Wa31. The eighth driving wire W32 includes an eighth held portion Wa32. The ninth driving wire W33 includes a ninth held portion Wa33.

In the present example, the first to ninth held portions (Wa11 to Wa33) each have the same shape.

The first to ninth driving wires (W11 to W33) each include a flexible wire body (line body, linear body) Wb. Here, the wire body Wb is a member through which a connected object can be pushed and pulled, and has stiffness of a certain degree. Meanwhile, the wire body Wb is a member capable of deforming from a linear shape so as to be able to bend the bending portion 12. Specifically, the first driving wire W11 includes a first wire body Wb11. The second driving wire W12 includes a second wire body Wb12. The third driving wire W13 includes a third wire body Wb13. The fourth driving wire W21 includes a fourth wire body Wb21. The fifth driving wire W22 includes a fifth wire body Wb22. The sixth driving wire W23 includes a sixth wire body Wb23. The seventh driving wire W31 includes a seventh wire body Wb31. The eighth driving wire W32 includes an eighth wire body Wb32. The ninth driving wire W33 includes a ninth wire body Wb33.

In the present example, the first to third wire bodies (Wb11 to Wb13) each have the same shape. The fourth to sixth wire bodies (Wb21 to Wb23) each have the same shape. The seventh to ninth wire bodies (Wb31 to Wb33) each have the same shape. In the present example, the first to ninth wire bodies (Wb11 to Wb33) each have the same shape except for the length thereof.

The first to ninth held portions (Wa11 to Wa33) are fixed to the first to ninth wire bodies (Wb11 to Wb33) at the near ends of the first to ninth wire bodies (Wb11 to Wb33).

The first to ninth driving wires (W11 to W33) are inserted in the bending portion 12 via a wire guide 17 and are fixed.

In the present example, the material of the wire body Wb of each of the first to ninth wire (W11 to W33) is metal. To be noted, the material of the wire body Wb of each of the first to ninth wires (W11 to W33) may be resin. The material of the wire body Wb of each of the first to ninth wires (W11 to W33) may include metal and resin.

Arbitrary one of the first to ninth driving wires (W11 to W33) can be referred to as a driving wire W. In the present example, the first to ninth driving wires (W11 to W33) each have the same shape except for the lengths of the first to ninth wire bodies (Wb11 to Wb33).

In the present example, the bending portion 12 is a tubular member having flexibility and having a pathway Ht for inserting the medical tool.

A plurality of wire holes for respectively allowing the first to ninth driving wires (W11 to W33) to pass through are provided in a wall surface of the bending portion 12. Specifically, a first wire hole Hw11, a second wire ole Hw12, and a third wire hole Hw13 are provided in the wall surface of the bending portion 12. Further, a fourth wire hole Hw21, a fifth wire hole Hw22, and a sixth wire hole Hw23 are provided in the wall surface of the bending portion 12. Further, a seventh wire hole Hw31, an eighth wire hole Hw32, and a ninth wire hole Hw33 are provided in the wall surface of the bending portion 12. The first to ninth wire holes Hw (Hw11 to Hw33) respectively correspond to the first to ninth driving wires (W11 to W33). The suffix numerals of the reference sign Hw indicate the numerals of the corresponding driving wires. For example, the first driving wire W11 is inserted in the first wire hole Hw11.

Arbitrary one of the first to ninth wire holes (Hw11 to Hw33) can be referred to as a wire hole Hw. In the present example, the first to ninth wire holes (Hw11 to Hw33) each have the same shape.

The bending portion 12 has an intermediate region 12a and a bending region 12b. The bending region 12b is disposed at the far end of the bending portion 12, and a first guide ring J1, a second guide ring J2, and a third guide ring J3 are disposed in the bending region 12b. The bending region 12b is a region where the degree and direction of bending of the bending portion 12 can be controlled by moving the first guide ring J1, the second guide ring J2, and the third guide ring J3 by the bending driving portion 13. FIG. 3B illustrates the bending portion 12 by omitting part thereof covering the first to third guide rings (J1 to J3).

In the present example, the bending portion 12 includes a plurality of auxiliary rings (not illustrated). In the bending region 12b, the first guide ring J1, the second guide ring J2, and the third guide ring J3 are fixed to the wall surface of the bending portion 12. In the present example, the plurality of auxiliary rings are disposed between the first guide ring J1 and the second guide ring J2 and between the second guide ring J2 and the third guide ring J3.

The medical tool is guided to the distal end of the catheter 11 by the pathway Ht, the first to third guide rings (J1 to J3), and the plurality of auxiliary rings.

The first to ninth driving wires (W11 to W33) are respectively fixed to the first to third guide rings (J1 to J3) through the intermediate region 12a.

Specifically, the first driving wire W11, the second driving wire W12, and the third driving wire W13 are fixed to the first guide ring J1. The fourth driving wire W21, the fifth driving wire W22, and the sixth driving wire W23 penetrate the first guide ring J1 and the plurality of auxiliary rings, and are fixed to the second guide ring J2. The seventh driving wire W31, the eighth driving wire W32, and the ninth driving wire W33 penetrate the first guide ring J1, the second guide ring J2, and the plurality of auxiliary rings, and are fixed to the third guide ring J3.

The medical apparatus 1 can bend the bending portion 12 toward a direction intersecting with the extending direction of the catheter 11 by driving the bending driving portion 13 by the wire driving portion 300. Specifically, by moving each of the first to ninth driving wires (W11 to W33) in the extending direction of the bending portion 12, the bending region 12b of the bending portion 12 can be bent in a direction intersecting with the extending direction via the first to third guide rings (J1 to J3).

The user can insert the catheter 11 to a target portion in the object Obj by using at least one of movement of the medical apparatus 1 moved manually or by the moving stage 2a and bending of the bending portion 12.

To be noted, although the bending portion 12 is bent by moving the first to third guide rings (J1 to J3) by the first to ninth driving wires (W11 to W33) in the present example, the present technique is not limited to this configuration. One or two of the first to third guide rings (J1 to J3) and driving wires fixed thereto may be omitted.

For example, the catheter 11 may have a configuration in which the seventh to ninth driving wires (W31 to W33) and the third guide ring J3 are provided and the first to sixth driving wires (W11 to W23) and the first and second guide rings (J1 to J2) are omitted. In addition, the catheter 11 may have a configuration in which the fourth to ninth driving wires (W21 to W33) and the second and third guide rings J2 and J3 are provided and the first to third driving wires (W11 to W13) and the first guide ring (J1) is omitted

In addition, the catheter 11 may have a configuration in which one guide ring is driven by two driving wires. Also in this case, the number of guide rings may be equal to or more than one.

### <Catheter Unit>

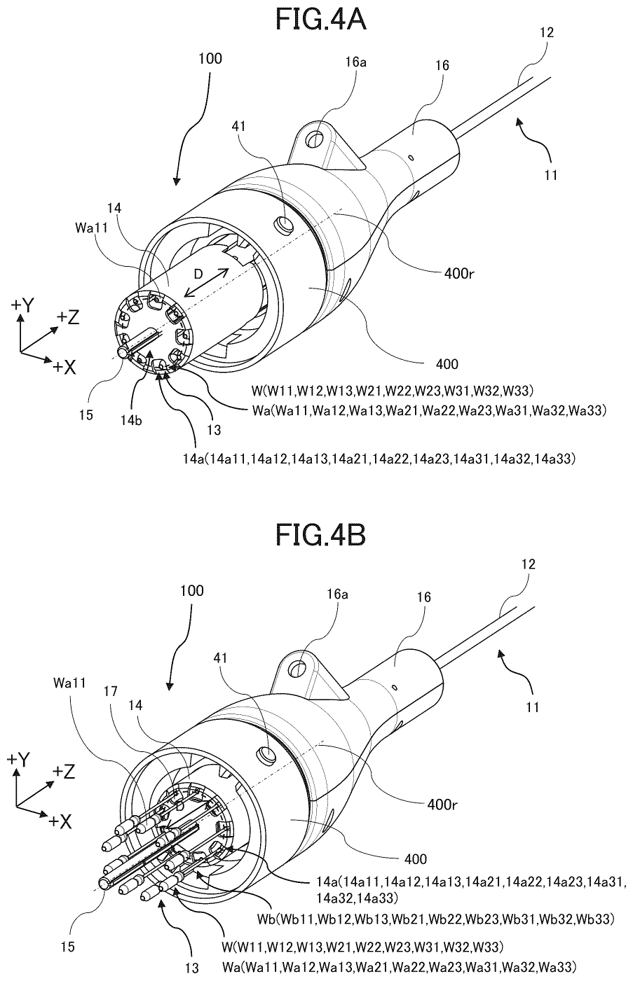

The catheter unit 100 will be described with reference to FIGS. 4A and 4B. FIGS. 4A and 4B are explanatory diagrams of the catheter unit 100. FIG. 4A is an explanatory diagram of the catheter unit 100 in a state in which a wire cover 14 that will be described later is at a covering position. FIG. 4B is an explanatory diagram of the catheter unit 100 in a state in which the wire cover 14 is at an exposing position.

The catheter unit 100 includes the bending portion 12, the catheter 11 including the bending driving portion 13, and a near-end cover 16 supporting the near end of the catheter 11. The catheter unit 100 includes a cover (wire cover) 14 for covering and protecting the first to ninth driving wires (W11 to W33) serving as a plurality of driving wires.

The catheter unit 100 is attachable to and detachable from the base unit 200 in an attachment/detachment direction D. The attachment direction of the catheter unit 100 to the base unit 200 and the detachment direction of the catheter unit 100 from the base unit 200 are parallel to the attachment/detachment direction D.

The near-end cover (frame body, bending portion casing, catheter casing) 16 is a cover covering a part of the catheter 11. The near-end cover 16 includes a tool hole 16a for inserting a medical tool into the pathway Ht of the bending portion 12.

The wire cover 14 is provided with a plurality of exposing holes (wire cover holes, cover holes) for allowing the first to ninth driving wires (W11 to W33) to pass through. The wire cover 14 is provided with a first exposing hole 14a11, a second exposing hole 14a 12, a third exposing hole 14a13, a fourth exposing hole 14a21, a fifth exposing hole 14a22, a sixth exposing hole 14a23, a seventh exposing hole 14a31, and an eighth exposing hole 14a32, and a ninth exposing hole 14a33. The first to ninth exposing holes (14a11 to 14a33) respectively correspond to the first to ninth driving wires (W11 to W33). The suffix numerals after the reference sign 14a indicate the numerals of the corresponding driving wires. For example, the first driving wire W11 is inserted in the first exposing hole 14a11.

Arbitrary one of the first to ninth exposing holes (14a11 to 14a33) can be referred to as an exposing hole 14a. In the present example, the first to ninth exposing holes (14a11 to 14a33) each have the same shape.

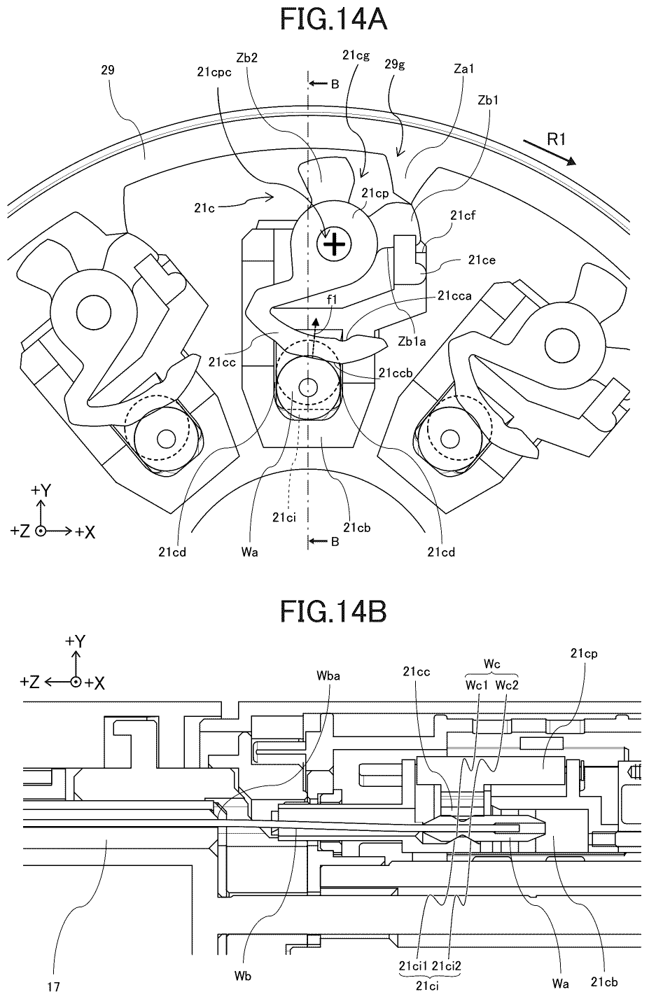

The wire cover 14 can move to a covering position (see FIG. 14A) covering the first to ninth driving wires (W11 to W33), and a cover retraction position (FIG. 14B) retracted from the covering position. The cover retraction position can be also referred to as an exposing position where the first to ninth driving wires (W11 to W33) are exposed.

Before attaching the catheter unit 100 to the base unit 200, the wire cover 14 is positioned at the covering position. When the catheter unit 100 is attached to the base unit 200, the wire cover 14 moves from the covering position to the exposing position in the attachment/detachment direction D.

When the wire cover 14 is at the exposing position, the first to ninth held portions (Wa11 to Wa33) of the first to ninth driving wires (W11 to W33) are exposed. As a result of this, the coupling between the bending driving portion 13 and a coupling device 21 that will be described later is allowed. When the wire cover 14 is at the exposing position, the first to ninth held portions (Wa11 to Wa33) of the first to ninth driving wires (W11 to W33) and part of the wire body Wb project from the first to ninth exposing holes (14a11 to 14a33). More specifically, the first to ninth held portions (Wa11 to Wa33) project from the first to ninth exposing holes (14a11 to 14a33) in an attachment direction Da that will be described later.

As illustrated in FIG. 4B, the first to ninth driving wires (W11 to W33) are each arranged along a circle (virtual circle) having a predetermined radius, and are each supported by the wire guide 17.

In the present example, the catheter unit 100 includes a key shaft (key, catheter-side key) 15. In the present example, the key shaft 15 extends in the attachment/detachment direction D. The wire cover 14 is provided with a shaft hole 14b through which the key shaft 15 passes. The key shaft 15 is capable of engaging with a key receiving portion 22 that will be described later. As a result of the key shaft 15 engaging with the key receiving portion 22, the movement of the catheter unit 100 with respect to the base unit 200 is limited to a predetermined range in a circumferential direction of the circle (virtual circle) on which the first to ninth driving wires (W11 to W33) are arranged.

In the present example, the first to ninth driving wires (W11 to W33) are disposed outside the key shaft 15 so as to surround the key shaft 15 as viewed in the attachment/detachment direction D. In other words, the key shaft 15 is disposed inside the circle (virtual circle) on which the first to ninth driving wires (W11 to W33) are arranged.

In the present example, the catheter unit 100 includes an operation portion 400. The operation portion 400 is configured to be movable (rotatable) with respect to the near-end cover 16 and the bending driving portion 13. The operation portion 400 is rotatable about a rotation shaft 400r. The rotation shaft 400r of the operation portion 400 extends in the attachment/detachment direction D.

The operation portion 400 is configured to be movable (rotatable) with respect to the base unit 200 in a state in which the catheter unit 100 is attached to the base unit 200. More specifically, the operation portion 400 is configured to be movable (rotatable) with respect to the base casing 200f, the wire driving portion 300, and the coupling device 21 that will be described later.

### <Base Unit>

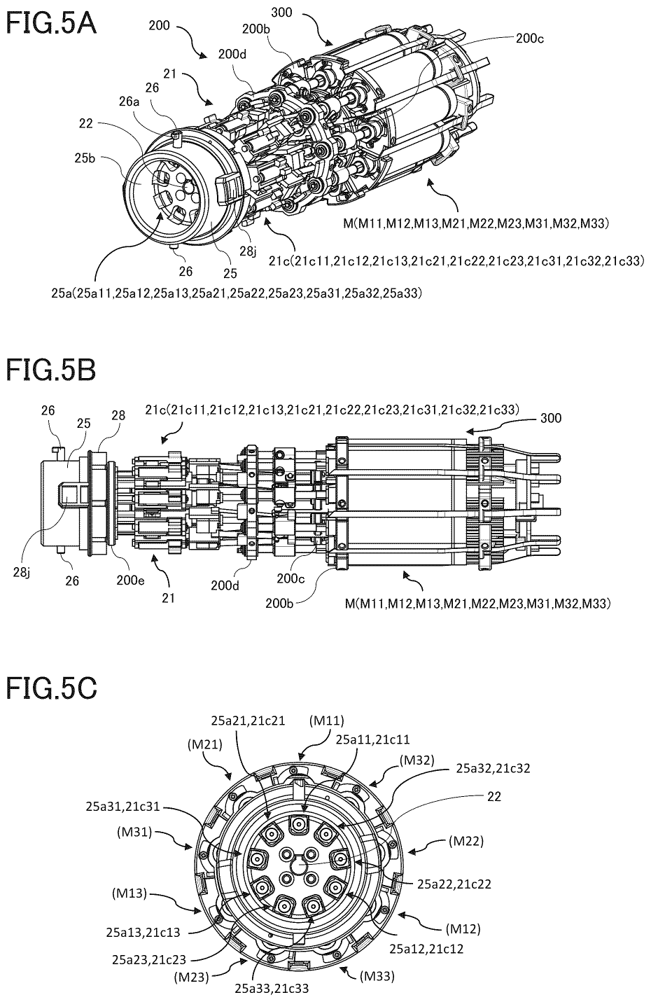

The base unit 200 and the wire driving portion 300 will be described with reference to FIGS. 5A to 5C. FIGS. 5A to 5C are explanatory diagrams of the base unit 200 and the wire driving portion 300. FIG. 5A is a perspective view illustrating an inner structure of the base unit 200. FIG. 5B is a side view illustrating the inner structure of the base unit 200. FIG. 5C is a diagram illustrating the base unit 200 as viewed in the attachment/detachment direction D.

As described above, the medical apparatus 1 includes the base unit 200 and the wire driving portion 300. In the present example, the wire driving portion 300 is accommodated in the base casing 200f, and is provided inside the base unit 200. In other words, the base unit 200 includes the wire driving portion 300.

The wire driving portion 300 includes a plurality of motors as a plurality of drive sources (actuators). In the present example, the wire driving portion 300 includes a first drive source M11, a second drive source M12, a third drive source M13, a fourth drive source M21, a fifth drive source M22, a sixth drive source M23, a seventh drive source M31, an eighth drive source M32, and a ninth drive source M33.

Arbitrary one of the first to ninth drive sources (M11 to M33) can be referred to as a drive source M. In the present example, the first to ninth drive sources (M11 to M33) each have the same configuration.

The base unit 200 includes the coupling device 21. The coupling device 21 is accommodated in the base casing 200f. The coupling device 21 is connected to the wire driving portion 300. The coupling device 21 includes a plurality of coupling portions. In the present example, the coupling device 21 includes a first coupling portion 21c11, a second coupling portion 21c12, a third coupling portion 21c13, a fourth coupling portion 21c21, a fifth coupling portion 21c22, a sixth coupling portion 21c23, a seventh coupling portion 21c31, an eighth coupling portion 21c32, and a ninth coupling portion 21c33.

Arbitrary one of the first to ninth coupling portions (21c11 to 21c33) can be referred to as a coupling portion 21c. In the present example, the first to ninth coupling portions (21c11 to 21c33) each have the same configuration.

The plurality of coupling portions are respectively connected to the plurality of drive sources, and are respectively driven by the plurality of drive sources. Specifically, the first coupling portion 21c11 is connected to the first drive source M11, and is driven by the first drive source M11. The second coupling portion 21c12 is connected to the second drive source M12, and is driven by the second drive source M12. The third coupling portion 21c13 is connected to the third drive source M13, and is driven by the third drive source M13. The fourth coupling portion 21c21 is connected to the fourth drive source M21, and is driven by the fourth drive source M21. The fifth coupling portion 21c22 is connected to the fifth drive source M22, and is driven by the fifth drive source M22. The sixth coupling portion 21c23 is connected to the sixth drive source M23, and is driven by the sixth drive source M23. The seventh coupling portion 21c31 is connected to the seventh drive source M31, and is driven by the seventh drive source M31. The eighth coupling portion 21c32 is connected to the eighth drive source M32, and is driven by the eighth drive source M32. The ninth coupling portion 21c33 is connected to the ninth drive source M33, and is driven by the ninth drive source M33.

As will be described later, the coupling device 21 is coupled to the bending driving portion 13 including the first to ninth driving wires (W11 to W33). The bending driving portion 13 receives the driving force of the wire driving portion 300 via the coupling device 21 and bends the bending portion 12.

The driving wire W is coupled to the coupling portion 21c via the held portion Wa. The plurality of driving wires are respectively coupled to the plurality of coupling portions.

Specifically, the first held portion Wa11 of the first driving wire W11 is coupled to the first coupling portion 21c11. The second held portion Wa12 of the second driving wire W12 is coupled to the second coupling portion 21c12. The third held portion Wa13 of the third driving wire W13 is coupled to the third coupling portion 21c13. The fourth held portion Wa21 of the fourth driving wire W21 is coupled to the fourth coupling portion 21c21. The fifth held portion Wa22 of the fifth driving wire W22 is coupled to the fifth coupling portion 21c22. The sixth held portion Wa23 of the sixth driving wire W23 is coupled to the sixth coupling portion 21c23. The seventh held portion Wa31 of the seventh driving wire W31 is coupled to the seventh coupling portion 21c31. The eighth held portion Wa32 of the eighth driving wire W32 is coupled to the eighth coupling portion 21c32. The ninth held portion Wa33 of the ninth driving wire W33 is coupled to the ninth coupling portion 21c33.

The base unit 200 includes a base frame 25. The base frame 25 is provided with a plurality of insertion holes for respectively allowing the first to ninth driving wires (W11 to W33) to pass through. The base frame 25 has a first insertion hole 25a11, a second insertion hole 25a12, a third insertion hole 25a13, a fourth insertion hole 25a21, a fifth insertion hole 25a22, a sixth insertion hole 25a23, a seventh insertion hole 25a31, an eighth insertion hole 25a32, and a ninth insertion hole 25a33. The first to ninth insertion holes (25a11 to 25a33) respectively correspond to the first to ninth driving wires (W11 to W33). The suffix numerals after the reference sign 25a indicate the numerals of the corresponding driving wires. For example, the first driving wire W11 is inserted in the first insertion hole 25a11.

Arbitrary one of the first to ninth insertion holes (25a11 to 25a33) can be referred to as an insertion hole 25a. In the present example, the first to ninth insertion holes (25a11 to 25a33) each have the same shape.

The base frame 25 is provided with an attachment opening 25b in which the wire cover 14 is inserted. The first to ninth insertion holes (25a11 to 25a33) are provided at a bottom portion of the attachment opening 25b.

Further, the base unit 200 includes a motor frame 200b, a first bearing frame 200c, a second bearing frame 200d, and a third bearing frame 200e. The motor frame 200b, the first bearing frame 200c, the second bearing frame 200d, and the third bearing frame 200e are coupled (fastened) to each other.

The base frame 25 includes a key receiving portion (key hole, base-side key, body-side key) 22 that receives the key shaft 15. As a result of the key shaft 15 and the key receiving portion 22 engaging with each other, the catheter unit 100 is prevented from being attached to the base unit 200 in a wrong phase.

As a result of the key shaft 15 and the key receiving portion 22 engaging with each other, the movement of the catheter unit 100 with respect to the base unit 200 is limited to a predetermined range in the circumferential direction of the circle (virtual circle) on which the first to ninth driving wires (W11 to W33) are arranged.

As a result, the first to ninth driving wires (W11 to W33) respectively engage with the corresponding ones of the first to ninth insertion holes (25a11 to 25a33) and the corresponding ones of the first to ninth coupling portions (21c11 to 21c33). In other words, the driving wire W is prevented from engaging with an insertion hole 25a different from the corresponding insertion hole 25a or a coupling portion 21c different from the corresponding coupling portion 21c.

The user can correctly couple the first to ninth driving wires (W11 to W33) respectively to the first to ninth coupling portions (21c11 to 21c33) by engaging the key shaft 15 and the key receiving portion 22 with each other. Therefore, the user can easily attach the catheter unit 100 to the base unit 200.

In the present example, the key shaft 15 includes a protrusion portion protruding in a direction intersecting with the attachment/detachment direction D, and the key receiving portion 22 has a recess portion in which the protrusion portion is inserted. In the circumferential direction, a position where the protrusion portion and the recess portion engage with each other is a position where the driving wire W engages with the corresponding insertion hole 25a and the corresponding coupling portion 21c.

To be noted, the key shaft 15 can be provided at one of the base unit 200 and the catheter unit 100, and the key receiving portion 22 can be provided at the other. For example, the key shaft 15 may be provided on the base unit 200 side, and the key receiving portion 22 may be provided on the catheter unit 100 side.

The base unit 200 includes a joint 28 including a joint engagement portion 28j. The base frame 25 includes a locking shaft 26 including a locking protrusion 26a. Functions of these will be described later.

<Coupling between Motors and Driving Wires>

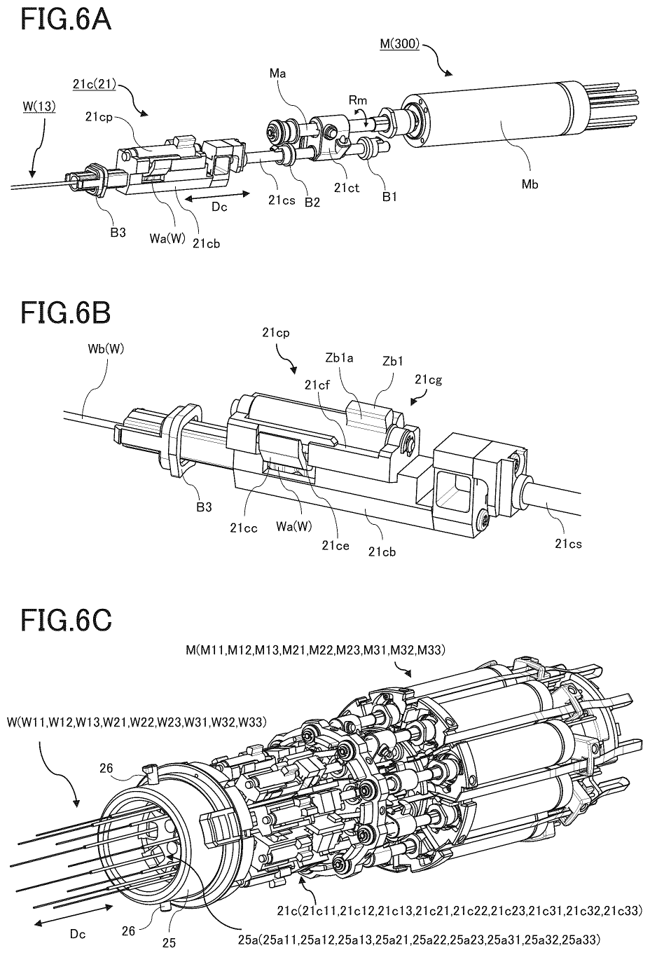

Coupling between the wire driving portion 300, the coupling device 21, and the bending driving portion 13 will be described with reference to FIGS. 6A to 6C.

FIGS. 6A to 6C are explanatory diagrams of the wire driving portion 300, the coupling device 21, and the bending driving portion 13. FIG. 6A is a perspective view of the drive source M, the coupling portion 21c, and the driving wire W. FIG. 6B is an enlarged view of the coupling portion 21c and the driving wire W. FIG. 6C is a perspective view illustrating the coupling between the wire driving portion 300, the coupling device 21, and the bending driving portion 13.

In the present example, the configurations of coupling respectively between the first to ninth driving wires (W11 to W33) and the first to ninth coupling portions (21c11 to 21c33) are the same. In addition, the configurations of respective connection between the first to ninth coupling portions (21c11 to 21c33) and the first to ninth drive sources (M11 to M33) are the same. Therefore, in the description below, the configurations of connection between these will be described by using one driving wire W, one coupling portion 21c, and one drive source M.

As illustrated in FIGS. 6A and 6B, the drive source M includes an output shaft Ma and a motor body Mb that rotates the output shaft Ma in a rotational direction Rm. A spiral groove is provided on the surface of the output shaft Ma. The output shaft Ma has a so-called screw shape. The motor body Mb is fixed to the motor frame 200b (FIGS. 5A and 5B).

The coupling portion 21c includes a tractor 21ct connected to the output shaft Ma and a tractor support shaft 21cs that supports the tractor 21ct. The tractor support shaft 21cs is connected to a coupling base 21cb.

The coupling portion 21c includes a first rotary body 21cp for pressing the held portion Wa of the driving wire W. The driving wire W is coupled to the coupling portion 21c through the insertion hole 25a (FIG. 6C) of the base frame 25. As will be described in detail later, as a result of the rotation of the first rotary body 21cp, a state (locked state, engaged state) in which the held portion Wa is fixed with respect to the coupling base 21cb and a state (released state) in which the fixation of the held portion Wa with respect to the coupling base 21cb is cancelled are switched.

The state in which the held portion Wa is fixed with respect to the coupling base 21cb is a state in which relative movement of the held portion Wa with respect to the coupling base 21cb in the longitudinal direction of the driving wire W is restricted. The state in which the fixation of the held portion Wa with respect to the coupling base 21cb is cancelled is a state in which relative movement of the held portion Wa with respect to the coupling base 21cb in the longitudinal direction is allowed.

The first rotary body 21cp includes a gear portion 21cg that engages with an internal gear 29 that will be described later, and a cam 21cc serving as a pressing portion for pressing the held portion Wa of the driving wire W. The cam 21cc is movable to a pressing position to press the held portion Wa to fix the held portion Wa with respect to the coupling base 21cb, and a retracted position to be retracted from the held portion Wa to cancel the fixation of the held portion Wa with respect to the coupling base 21cb. That is, as a result of movement of the cam 21cc, the held portion Wa can be fixed with respect to the coupling base 21cb, and the fixation can be cancelled.

The coupling portion 21c is supported by a first bearing B1, a second bearing B2, and a third bearing B3. The first bearing B1 is supported by a first bearing frame 200c of the base unit 200. The second bearing B2 is supported by a second bearing frame 200d of the base unit 200. The third bearing B3 is supported by a third bearing frame 200e of the base unit 200. Therefore, when the output shaft Ma rotates in the rotational direction Rm, rotation of the coupling portion 21c about the output shaft Ma is restricted. To be noted, the first bearing B1, the second bearing B2, and the third bearing B3 are each provided for each of the first to ninth coupling portions (21c11 to 21c33).

Since the rotation of the coupling portion 21c about the output shaft Ma is restricted, a force in the rotational axis direction of the output shaft Ma acts on the tractor 21ct due to the spiral groove of the output shaft Ma when the output shaft Ma rotates. As a result, the coupling portion 21c moves in the rotational axis direction (Dc direction) of the output shaft Ma. The rotational axis direction of the output shaft Ma is substantially parallel to the extending direction (longitudinal direction) of the driving wire W in the coupling portion 21c. As a result of the coupling portion 21c moving to one side or the other side in the Dc direction, the driving wire W moves to the one side or the other side, and the bending portion 12 bends.

That is, the output shaft Ma and the tractor 21ct constitute a so-called feed screw that converts the rotational motion transmitted from the drive source M into a linear motion by the screw. In the present example, the output shaft Ma and the tractor 21ct are a slide screw, but may be a ball screw.

As illustrated in FIG. 6C, by attaching the catheter unit 100 to the base unit 200, the first to ninth driving wires (W11 to W33) are respectively coupled to the first to ninth coupling portions (21c11 to 21c33).

The control portion 3 can control the first to ninth drive sources (M11 to M33) independently from each other. That is, an arbitrary drive source among the first to ninth drive sources (M11 to M33) can be independently operated or stopped regardless of whether or not the other drive sources are stopped. In other words, the control portion 3 can control the first to ninth driving wires (W11 to W33) independently from each other. As a result, the first to third guide rings (J1 to J3) are controlled independently from each other, and the bending region 12b of the bending portion 12 can be bent in an arbitrary direction.

### <Attachment of Catheter Unit>

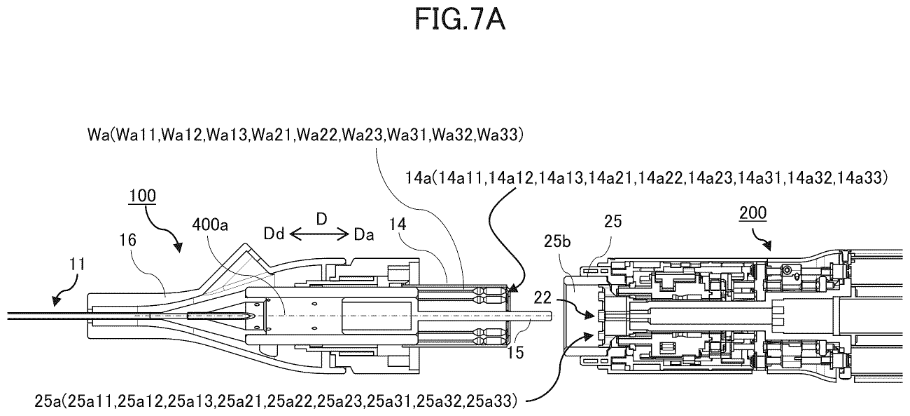

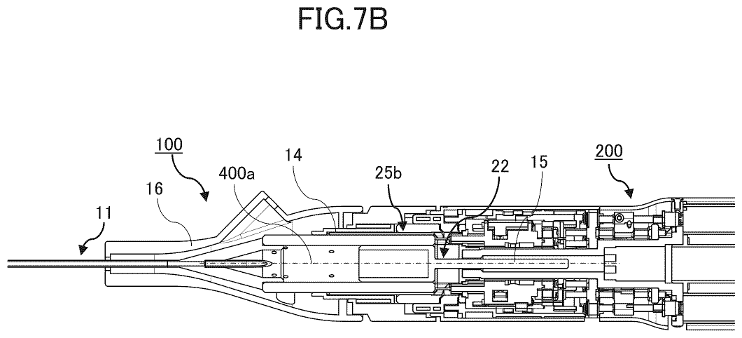

An operation of attaching the catheter unit 100 to the base unit 200 will be described with reference to FIGS. 7A and 7B.

FIGS. 7A and 7B are explanatory diagrams of attachment of the catheter unit 100. FIG. 7A is a diagram before the catheter unit 100 is attached to the base unit 200. FIG. 7B is a diagram after the catheter unit 100 is attached to the base unit 200.

In the present example, the attachment/detachment direction D of the catheter unit 100 is the same as the direction of the rotation shaft 400r of the operation portion 400. In addition, the attachment/detachment direction D is the same as the extending direction (longitudinal direction) of each driving wire W in the coupling portion 21c. In the attachment/detachment direction D, a direction in which the catheter unit 100 is attached to the base unit 200 will be referred to as an attachment direction Da. In the attachment/detachment direction D, a direction in which the catheter unit 100 is detached from the base unit 200 (direction opposite to the attachment direction Da) will be referred to as a detachment direction Dd.

As illustrated in FIG. 7A, in a state before the catheter unit 100 is attached to the base unit 200, the wire cover 14 is positioned at the covering position. At this time, the wire cover 14 covers the first to ninth driving wires (W11 to W33) such that the first to ninth held portions (Wa11 to Wa33) do not project from the first to ninth exposing holes (14a11 to 14a33) of the wire cover 14.

When the key shaft 15 and the key receiving portion 22 engage with each other and the catheter unit 100 moves in the attachment direction Da with respect to the base unit 200, the catheter unit 100 is attached to the base unit 200. By attaching the catheter unit 100 to the base unit 200, the wire cover 14 moves to the exposing position. In the present example, the wire cover 14 moves from the covering position to the exposing position by abutting the base frame 25 (see FIG. 7B).

More specifically, when attaching the catheter unit 100, the wire cover 14 stops by abutting the base frame 25. In this state, by moving the catheter unit 100 in the attachment direction Da, the wire cover 14 relatively moves with respect to parts other than the wire cover 14 in the catheter unit 100. As a result, the wire cover 14 moves from the covering position to the exposing position.

While the wire cover 14 moves from the covering position to the exposing position, the held portion Wa of the driving wire W projects from the exposing hole 14a of the wire cover 14, and is inserted in the insertion hole 25a of the base frame 25. Then, the held portion Wa is engaged with the coupling base 21cb of the coupling portion 21c (see FIG. 6B).

In a state in which the catheter unit 100 is merely attached to the base unit 200, the catheter unit 100 can be detached from the base unit 200 by moving the catheter unit 100 in the detachment direction Dd with respect to the base unit 200. In addition, as will be described later, in a state in which the catheter unit 100 is merely attached to the base unit 200, the driving wire W and the coupling portion 21c are not fixed.

Separation (detachment) of the catheter unit 100 from the base unit 200 can be prevented by operating the operation portion 400 in a state in which the catheter unit 100 is attached to the base unit 200. Further, by operating the operation portion 400 in a state in which the catheter unit 100 is attached to the base unit 200, the driving wire W is fixed to the coupling portion 21c and the bending driving portion 13 is coupled to the wire driving portion 300 via the coupling device 21.

### <Coupling of Bending Driving Portion and Cancellation of Coupling>

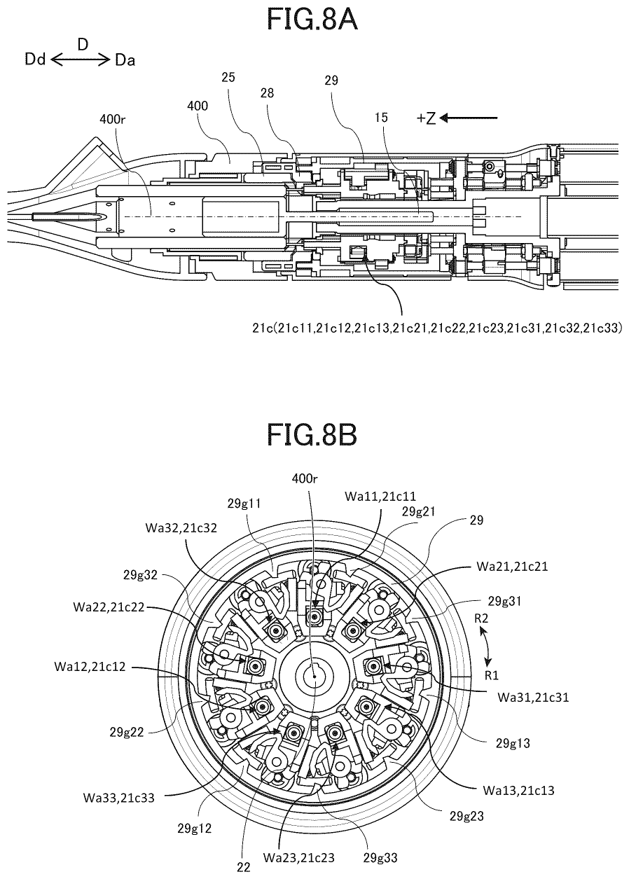

Elements for fixing and releasing the driving wire W with respect to the coupling portion 21c (elements for coupling the bending driving portion 13 by the coupling device 21 and cancelling the coupling) will be described with reference to FIGS. 8A, 8B, 9, 10A, 10B, 11, 12, 13, 14A, and 14B.

FIGS. 8A and 8B are diagrams for describing the coupling between the catheter unit 100 and the base unit 200. FIG. 8A is a section view of the catheter unit 100 and the base unit 200. FIG. 8A is a section view of the catheter unit 100 and the base unit 200 taken along the rotation shaft 400r. FIG. 8B is a section view of the base unit 200. It is a section view of the base unit 200 taken along a virtual plane passing through the coupling portion 21c and orthogonal to the rotation shaft 400r.

In FIG. 8A and each diagram thereafter, the axial direction of the rotation shaft 400r of the operation portion 400 is illustrated as a Z direction. In the present example, the Z direction is substantially parallel to the attachment/detachment direction D of the catheter unit 100 and the extending direction (longitudinal direction) of each driving wire W in the coupling portion 21c. In addition, directions each orthogonal to the Z direction and orthogonal to each other are illustrated as an X direction and a Y direction. If necessary, one side and the other side in each direction will be distinguished by adding a + or − sign on the basis of the direction of illustrated arrows. For example, the +Z direction side (+Z side) is a side on which the catheter unit 100 is positioned with respect to the base unit 200, and the −Z direction side (−Z side) is a side opposite thereto (side on which the base unit 200 is positioned with respect to the catheter unit 100).

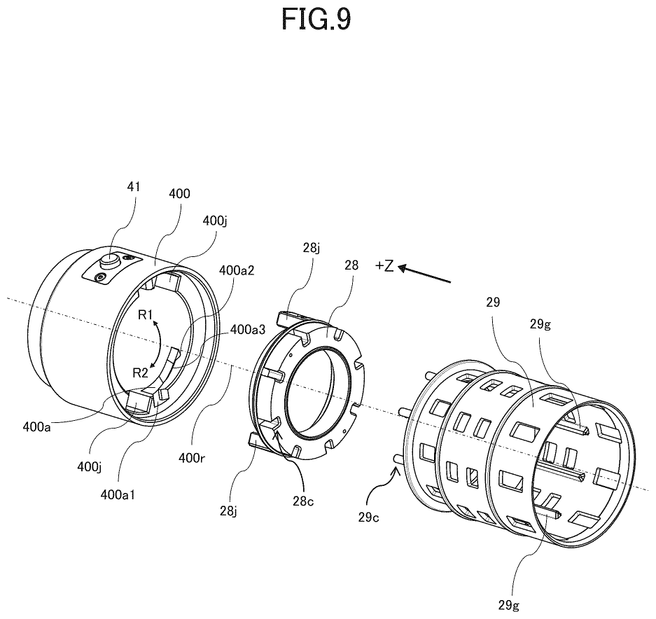

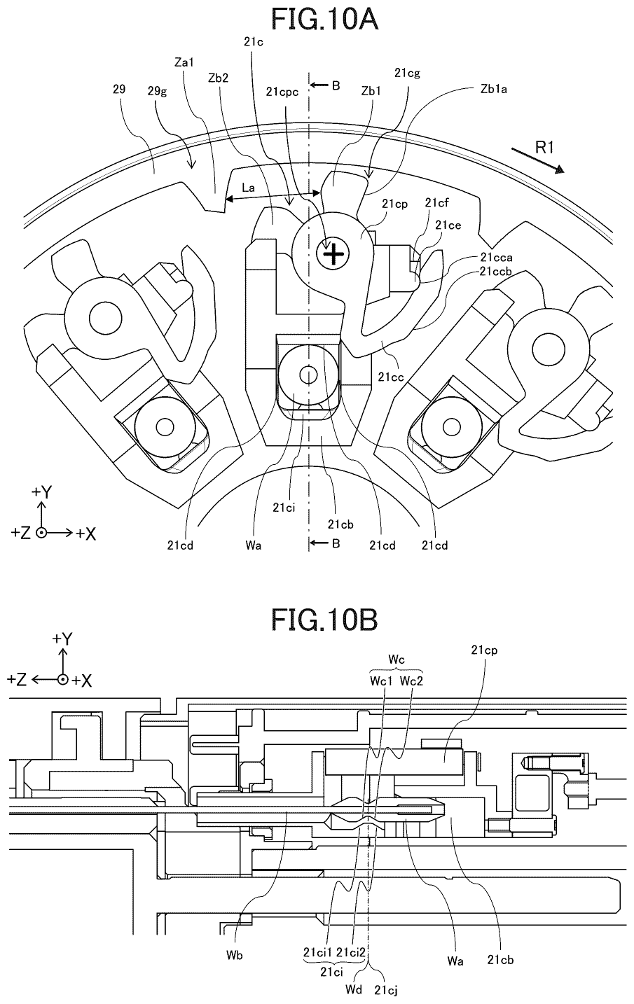

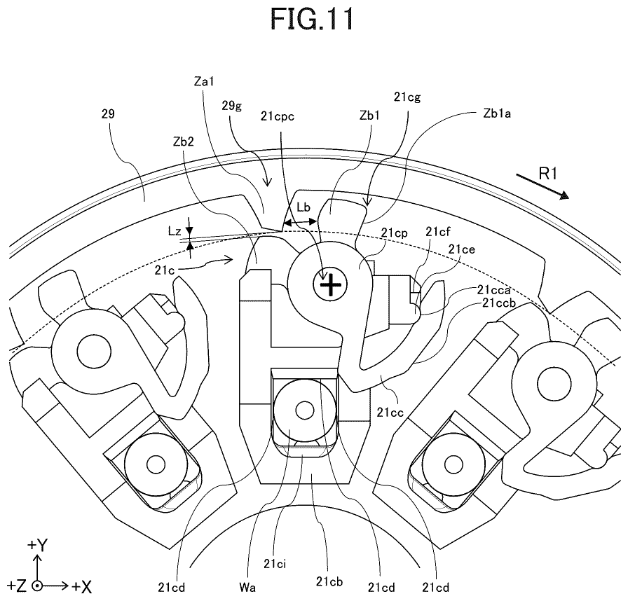

FIG. 9 is an exploded view for describing the coupling between the catheter unit 100 and the base unit 200. FIGS. 10A, 10B, 11, 12, 13, 14A, and 14B are diagrams for describing the fixation of the driving wire W by the coupling portion 21c.

As illustrated in FIGS. 8A and 9, the base unit 200 includes the joint 28 (intermediate member, second transmission member), and the internal gear 29 serving as a moving gear (interlocking gear, transmission member, first transmission member) that moves in an interlocked manner with the operation portion 400 via the joint 28.

The joint 28 includes a plurality of transmission portions 28c, and the internal gear 29 includes a plurality of transmitted portions 29c. The plurality of transmission portions 28c are engaged with the plurality of transmitted portions 29c, and in the case where the joint 28 rotates, the rotation of the joint 28 is transmitted to the internal gear 29.

When the catheter unit 100 is attached to the base unit 200, an engagement portion 400j provided on the operation portion 400 engages with a joint engagement portion 28j of the joint 28. In the case where the operation portion 400 rotates about the rotation shaft 400r, the rotation of the operation portion 400 is transmitted to the internal gear 29 via the joint 28. That is, in a state in which the catheter unit 100 is attached to the base unit 200, the operation portion 400, the joint 28, and the internal gear 29 integrally rotate in the same direction.

The internal gear 29 includes a plurality of tooth portions for switching the first to ninth coupling portions (21c11 to 21c33) between a state of respectively fixing the first to ninth driving wires (W11 to W33) and a state of respectively releasing the first to ninth driving wires (W11 to W33). The plurality of tooth portions (acting portions, switching gear portions) of the internal gear 29 respectively engage with the gear portions 21cg of the first rotary bodies 21cp of the first to ninth coupling portions (21c11 to 21c33).

Specifically, the internal gear 29 of the present example includes a first tooth portion 29g11, a second tooth portion 29g12, a third tooth portion 29g13, a fourth tooth portion 29g21, a fifth tooth portion 29g22, a sixth tooth portion 29g23, a seventh tooth portion 29g31, an eighth tooth portion 29g32, and a ninth tooth portion 29g33 (FIG. 8B). The first to ninth tooth portions (29g11 to 29g33) are formed with gaps therebetween on a circle (virtual circle) centered on the rotation shaft 400r.

The first tooth portion 29g11 engages with the gear portion 21cg of the first coupling portion 21c11. The second tooth portion 29g12 engages with the gear portion 21cg of the second coupling portion 21c12. The third tooth portion 29g13 engages with the gear portion 21cg of the third coupling portion 21c13. The fourth tooth portion 29g21 engages with the gear portion 21cg of the fourth coupling portion 21c21. The fifth tooth portion 29g22 engages with the gear portion 21cg of the fifth coupling portion 21c22. The sixth tooth portion 29g23 engages with the gear portion 21cg of the sixth coupling portion 21c23. The seventh tooth portion 29g31 engages with the gear portion 21cg of the seventh coupling portion 21c31. The eighth tooth portion 29g32 engages with the gear portion 21cg of the eighth coupling portion 21c32. The ninth tooth portion 29g33 engages with the gear portion 21cg of the ninth coupling portion 21c33.

Arbitrary one of the first to ninth tooth portions (29g11 to 29g33) can be referred to as a tooth portion 29g. In the present example, the first to ninth tooth portions (29g11 to 29g33) each have the same configuration.

In the present example, configurations in which the first to ninth driving wires (W11 to W33) are respectively coupled to the first to ninth coupling portions (21c11 to 21c33) are the same. In addition, configurations in which the first to ninth coupling portions (21c11 to 21c33) are respectively connected to the first to ninth tooth portions (29g11 to 29g33) are the same. Therefore, in the description below, by using one driving wire W, one coupling portion 21c, and one tooth portion 29g, a configuration in which these are connected will be described below.

According to the configuration of the joint 28 and the internal gear 29 described above, by rotating the operation portion 400, the internal gear 29 rotates. As a result of the internal gear 29 rotating, the first to ninth coupling portions (21c11 to 21c33) each operate. That is, in each of the first to ninth coupling portions (21c11 to 21c33), as a result of the gear portion 21cg being moved by the internal gear 29, the first rotary body 21cp rotates, and thus the cam 21cc moves to a pressing position and a retracted position that will be described later.

The operation portion 400 can move to a fixation position (locking position) and a detachment position in a state in which the catheter unit 100 is attached to the base unit 200. In addition, as will be described later, the operation portion 400 can move to a release position in a state in which the catheter unit 100 is attached to the base unit 200. The release position is positioned between the fixation position and the detachment position in the rotational direction of the operation portion 400 (rotational direction about the rotation shaft 400r). In a state in which the operation portion 400 is at the detachment position, the catheter unit 100 is attached to the base unit 200.

In a state immediately after the catheter unit 100 is attached to the base unit 200, the fixation (lock) of the driving wire W to the coupling portion 21c is cancelled. This state will be referred to as a released state of the coupling portion 21c. To be noted, a state in which the driving wire W is fixed (locked) to the coupling portion 21c will be referred to as a locked state of the coupling portion 21c.

The operation at the time of fixing the driving wire W to the coupling portion 21c will be described with reference to FIGS. 10A, 10B, 11, 12, 13, 14A, 14B, and 15. To be noted, FIGS. 10A, 11 to 13, 14A, and 15 are section views of the catheter unit 100 and the base unit 200 in a virtual plane orthogonal to the Z direction. In FIGS. 10A, 11 to 13, 14A, and 15, for a pair of interest of the driving wire W and the coupling portion 21c, the side on which the driving wire W is positioned with respect to the rotation shaft 400r of the operation portion 400 is set as the +Y direction. In addition, FIGS. 10B and 14B are section views of the catheter unit 100 and the base unit 200 in the Y-Z plane passing through the pair of interest of the driving wire W and the coupling portion 21c.

In a state after the catheter unit 100 is attached to the base unit 200 and before the operation portion 400 is operated, the catheter unit 100 can be detached from the base unit 200. A state in which the catheter unit 100 can be detached from the base unit 200 will be referred to as a detachable state. FIGS. 10A and 10B are diagrams illustrating a state of the internal gear 29 and the coupling portion 21c in the detachable state. FIG. 10A illustrates the positional relationship of the internal gear 29 and the coupling portion 21c in a state in which the operation portion 400 is positioned at the detachment position. FIG. 10B is a section view of the catheter unit 100 and the base unit 200 taken along a line B-B of FIG. 10A.

The coupling base 21cb includes a cam holding portion 21ce (see also FIG. 6B), and a plurality of rod support surfaces 21cd that support the held portion Wa of the driving wire W. The cam 21cc provided at the first rotary body 21cp includes a holding surface 21cca and a pressing surface 21ccb. The plurality of rod support surfaces 21cd support the held portion Wa while restricting the movement of the held portion Wa except for a direction (−Y direction in the drawings) in which the held portion Wa is engaged with the coupling base 21cb among directions intersecting with the Z direction (longitudinal direction of the driving wire W). That is, the plurality of rod support surfaces 21cd form a recess shape of an approximate C shape opening on the −Y direction side (the protrusion portion 21ci side in the coupling base 21cb that will be described later) in a state viewed in the Z direction. In addition, the plurality of rod support surfaces 21cd extend in the Z direction to form a space where the held portion Wa enters the internal space of the coupling portion 21c when attaching the catheter unit 100 to the base unit 200.

As illustrated in FIG. 10A, in the attachable state (state in which the operation portion 400 is at the detachment position), the first rotary body 21cp is held at a position where the holding surface 21cca is engaged with the cam holding portion 21ce of the coupling base 21cb. Specifically, the holding surface 21cca is a recess portion that the cam holding portion 21ce is fit in, and since separation of the cam holding portion 21ce from the holding surface 21cca is restricted by the elasticity of the cam 21cc, the rotation of the first rotary body 21cp is prevented. In addition, a tooth Za1 of the internal gear 29 and a tooth Zb1 of the gear portion 21cg are stopped at positions with a clearance of a distance La therebetween.

In the rotational direction of the operation portion 400, a direction in which the operation portion 400 moves from the detachment position to the release position and the fixation position will be referred to as a locking direction R1 (fixing direction), and a direction in which the operation portion 400 moves from the fixation position to the release position and the detachment position will be referred to as a releasing direction R2 (see also FIG. 8B). The operation portion 400 rotates in the releasing direction R2 from the release position and thus moves to the detachment position. The operation portion 400 rotates in the locking direction R1 from the release position and thus moves to the fixation position.

In a state in which the catheter unit 100 is attached to the base unit 200 and the operation portion 400 is at the detachment position, the coupling portion 21c is in the released state, and the fixation of the driving wire W by the coupling portion 21c is cancelled. At this time, the held portion Wa is supported by the rod support surfaces 21cd provided on the coupling base 21cb, but the −Y direction side in FIG. 10A is not supported by the rod support surfaces 21cd. As a result of this, the held portion Wa is supported to be movable in only the −Y direction.

As illustrated in FIG. 10B, the held portion Wa includes a recess portion Wc, and the coupling base 21cb includes a protrusion portion 21ci capable of fitting in (engaging with) the recess portion Wc. The protrusion portion 21ci is a protrusion shape protruding in a direction intersecting with the extending direction (longitudinal direction, Z direction) of the driving wire W. The recess portion Wc is a recess shape recessed in a direction intersecting with the extending direction (longitudinal direction, Z direction) of the driving wire W.

In a state in which the protrusion portion 21ci is fitted in the recess portion Wc, the held portion Wa and the coupling base 21cb do not relatively move in the Z direction, and the held portion Wa integrally moves with the coupling base 21cb. That is, the held portion Wa and the coupling base 21cb are configured to take, by fitting the protrusion portion 21ci in the recess portion Wc, an engaged state in which these are engaged with each other such that the driving force of the drive source M is transmittable to the driving wire W. To be noted, the locked state of the coupling portion 21c in the present example indicates that the cam 21cc is positioned at the pressing position and the held portion Wa and the coupling base 21cb are in the engaged state.

When the operation portion 400 is at the detachment position, the cam 21cc is at the retracted position with a clearance from the held portion Wa. That is, as illustrated in FIG. 10B, the recess portion Wc of the held portion Wa is at a position separated from the protrusion portion 21ci of the coupling base 21cb in the +Y direction. That is, the fixation of the held portion Wa is cancelled (non-engaged state of the held portion Wa and the coupling base 21cb). Therefore, in the case where the catheter unit 100 is moved in the detachment direction Dd with respect to the base unit 200 in the state in which the operation portion 400 is at the detachment position, the held portion Wa can be pulled out from the coupling base 21cb.

FIG. 11 is a diagram illustrating a state of the internal gear 29 and the coupling portion 21c when the operation portion 400 is rotated in the locking direction R1 from the detachment position to the release position. FIG. 11 illustrates a state of the internal gear 29 and the coupling portion 21c in the state in which the operation portion 400 is at the release position.

When the operation portion 400 is rotated in the locking direction R1 from the detachment position (FIG. 10A, FIG. 10B), the internal gear 29 rotates in the locking direction R1 (clockwise in the drawing) in an interlocked manner with the operation portion 400. Thus, the operation portion 400 moves to the release position.

To be noted, even in the case where the operation portion 400 is rotated, since the key shaft 15 is engaged with the key receiving portion 22, rotation of the entirety (excluding the operation portion 400) of the catheter unit 100 with respect to the base unit 200 is restricted. That is, in a state in which a part excluding the operation portion 400 of the catheter unit 100 and the base unit 200 are stopped, the operation portion 400 is rotatable with respect to these.

As a result of the internal gear 29 rotating clockwise in the drawing, the clearance between the tooth Za1 of the internal gear 29 and the tooth Zb1 (first tooth) of the gear portion 21cg reduces from the distance La to a distance Lb, but the contact with the tooth Zb1 has not happened. Therefore, the cam 21cc of the first rotary body 21cp is positioned at the retracted position that is the same as in FIG. 10A. That is, even when the operation portion 400 moves from the detachment position to the release position, the cam 21cc stays at the retracted position. In addition, the coupling portion 21c is kept at the same state (released state) as the state illustrated in FIG. 10A.

A tooth Zb2 (second tooth) of the gear portion 21cg is disposed at a position with a clearance of a distance Lz from a tooth tip circle (dotted line) of a tooth portion 29g of the internal gear 29. Therefore, the internal gear 29 is capable of rotating without interfering with the tooth Zb2.

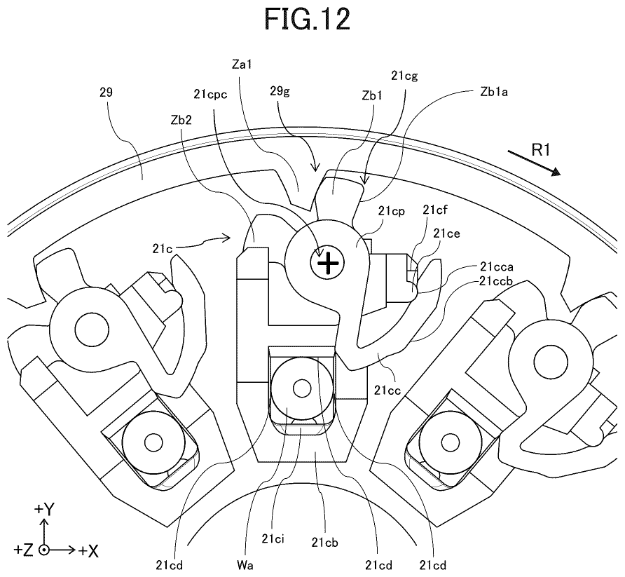

When the operation portion 400 is further rotated in the locking direction R1 from the state illustrated in FIG. 11, the internal gear 29 further rotates clockwise in the drawing. The state of the internal gear 29 and the coupling portion 21c at this time is illustrated in FIG. 12.

FIG. 12 is a diagram illustrating a state of the internal gear 29 and the coupling portion 21c when the operation portion 400 is rotated in the locking direction R1 from the release position. When the operation portion 400 is rotated in the locking direction R1 from the release position, the tooth Za1 of the internal gear 29 and the tooth Zb1 of the gear portion 21cg come into contact with each other. On the other hand, the cam 21cc is still positioned at the retracted position, and the coupling portion 21c is kept at the same released state as the state illustrated in FIGS. 10 and 11.

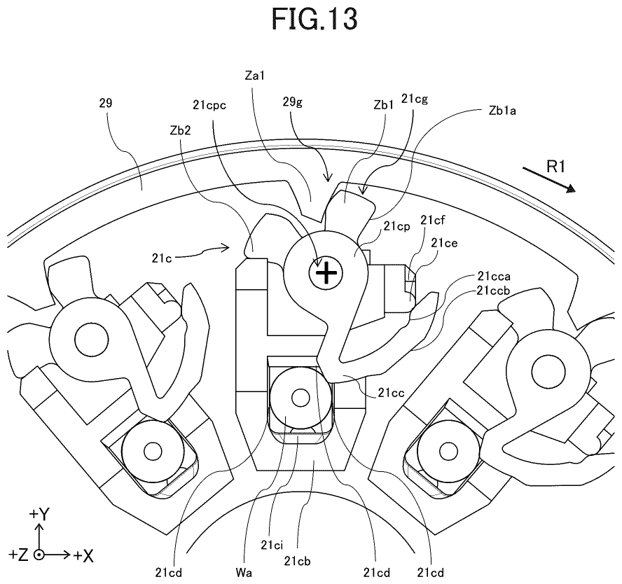

FIG. 13 is a diagram illustrating the state of the internal gear 29 and the coupling portion 21c when the operation portion 400 is further rotated in the locking direction R1 from the state of FIG. 12. As a result of further rotating the operation portion 400 in the locking direction R1 from the state of FIG. 12, the internal gear 29 further rotates clockwise in the drawing.

As a result of the internal gear 29 moving from the state of FIG. 12 to the state of FIG. 13, the internal gear 29 presses the gear portion 21cg of the first rotary body 21cp, and rotates the first rotary body 21cp clockwise in the drawing. When the first rotary body 21cp rotates, the engagement between the holding surface 21cca of the cam 21cc and the cam holding portion 21ce of the coupling base 21cb is cancelled. That is, the internal gear 29 rotates the first rotary body 21cp against the elastic force of the cam 21cc such that the holding surface 21cca is separated from the cam holding portion 21ce. Then, the cam 21cc of the first rotary body 21cp comes into contact with the held portion Wa, and the held portion Wa starts receiving force from the cam 21cc. That is, the cam 21cc starts moving from the retracted position to the pressing position.

FIG. 14A is a diagram illustrating the state of the internal gear 29 and the coupling portion 21c when the operation portion 400 is rotated in the locking direction R1 from the state of FIG. 13. When the operation portion 400 is further rotated in the locking direction R1 from the state of FIG. 13, the internal gear 29 is further rotated clockwise in the drawing.

As a result of the internal gear 29 moving from the state of FIG. 13 to the state of FIG. 14A, the internal gear 29 further rotates the gear portion 21cg clockwise in the drawing. The rotational trajectory of the pressing surface 21ccb when the gear portion 21cg rotates overlaps with the position of the held portion Wa (dotted line) in the state of FIG. 13. Therefore, when the gear portion 21cg rotates, the pressing surface 21ccb rotates while interfering with the held portion Wa (while pressing the held portion Wa). Here, the held portion Wa is supported in a state in which the held portion Wa is movable in the −Y direction and the movement thereof in other directions is restricted by the rod support surfaces 21cd provided on the coupling base 21cb. Therefore, the held portion Wa moves in the −Y direction by being pressed by the pressing surface 21ccb.

FIG. 14B is a section view of the catheter unit 100 and the base unit 200 taken along the line B-B of FIG. 14A, and illustrates the state of the internal gear 29 and the coupling portion 21c. When the held portion Wa moves in the −Y direction, the wire body Wb that is a flexible member supported by the wire guide 17 elastically deforms. Specifically, the wire body Wb in a cantilever beam state with a wire body support end Wba supported by the wire guide 17 warps such that the held portion Wa on the distal end side is displaced in the −Y direction. To be noted, in FIG. 14A, a circle of a broken line indicates the position of the held portion Wa before being pressed by the cam 21cc (see FIG. 10A).

In addition, when the held portion Wa moves in the −Y direction, the recess portion Wc provided on the held portion Wa and the protrusion portion 21ci provided on the coupling base 21cb engage with each other. As a result of this, a state in which the held portion Wa is relatively unmovable in the Z direction with respect to the coupling base 21cb, that is, a locked state of the coupling portion 21c is taken. In other words, the cam 21cc presses the held portion Wa so as to fix the held portion Wa with respect to the coupling base 21cb.

The engagement shape of the held portion Wa and the coupling base 21cb in the present example will be described in detail. As illustrated in FIG. 14B, the protrusion portion 21ci of the coupling base 21cb includes a first inclined surface 21ci1 that is a surface on the +Z direction side and a second inclined surface 21ci2 that is a surface on the −Z direction side. The recess portion Wc of the held portion Wa includes a first inclined surface Wc1 that is a surface on the +Z direction side and a second inclined surface Wc2 that is a surface on the −Z direction side.

The first inclined surface 21ci1 of the coupling base 21cb is a surface intersecting with the Z direction such that the normal direction thereof directs to the +Z direction side with respect to the Z direction. The second inclined surface 21ci2 of the coupling base 21cb is a surface intersecting with the Z direction such that the normal direction thereof directs to the −Z direction side with respect to the Z direction. The first inclined surface Wc1 of the held portion Wa is a surface intersecting with the Z direction such that the normal direction thereof directs to the −Z direction side with respect to the Z direction. The second inclined surface Wc2 of the held portion Wa is a surface intersecting with the Z direction such that the normal direction thereof directs to the +Z direction side with respect to the Z direction. To be noted, the recess portion Wc of the held portion Wa is a groove shape formed in an annular shape at an outer peripheral portion of the held portion Wa having an approximate columnar shape, and the first inclined surface Wc1 and the second inclined surface Wc2 are curved surfaces of a conical shape centered on the axis of the driving wire W.

In the engaged state of the coupling base 21cb (first member) and the held portion Wa (second member), the first inclined surface 21ci1 of the protrusion portion 21ci abuts the first inclined surface Wc1 of the recess portion Wc, and the second inclined surface 21ci2 of the protrusion portion 21ci abuts the second inclined surface Wc2 of the recess portion Wc. In the engaged state, in the case where the coupling base 21cb is driven in the +Z direction by the driving force of the drive source M, the first inclined surface 21ci1 (first abutting portion) of the coupling base 21cb presses the first inclined surface Wc1 (first abutted portion) of the held portion Wa in the +Z direction. As a result of this, the held portion Wa moves in the +Z direction together with the coupling base 21cb. In addition, in the engaged state, in the case where the coupling base 21cb is driven in the −Z direction by the driving force of the drive source M, the second inclined surface 21ci2 (second abutting portion) of the coupling base 21cb presses the second inclined surface Wc2 (second abutted portion) of the held portion Wa in the −Z direction. As a result of this, the held portion Wa moves in the −Z direction together with the coupling base 21cb. As described above, in the engaged state of the coupling base 21cb (first member) and the held portion Wa (second member), the coupling base 21cb and the held portion Wa integrally move in the +Z direction and the −Z direction.

The first inclined surface Wc1 of the held portion Wa is an example of a first abutted portion capable of receiving a force toward one side (+Z direction) in the longitudinal direction of the driving wire W from the coupling base 21cb by being abutted by the first inclined surface 21ci1 serving as a first abutting portion. In addition, the second inclined surface Wc2 of the held portion Wa is an example of a second abutted portion capable of receiving a force toward the other side (−Z direction) in the longitudinal direction of the driving wire W from the coupling base 21cb by being abutted by the second inclined surface 21ci2 serving as a second abutting portion. In the present example, since the driving force of the drive source M is transmitted to the driving wire W by mechanical engagement (physical contact) between the coupling base 21cb (first member) and the held portion Wa (second member), drive transmission of high reliability can be realized at a low cost.

Incidentally, the rotational trajectory of the pressing surface 21ccb of the cam 21cc overlaps also with the position of the held portion Wa in the locked state. That is, in the locked state, the cam 21cc is elastically deformed by an amount by which the rotational trajectory of the pressing surface 21ccb interferes with the held portion Wa. Therefore, in the locked state, the pressing surface 21ccb presses the held portion Wa by the elastic force of the cam 21cc, and thus the held portion Wa is strongly fixed to the coupling base 21cb.

When the tooth Za1 of the internal gear 29 is separated from the tooth Zb1 of the gear portion 21cg, the transmission of the driving force from the internal gear 29 to the gear portion 21cg is finished. At this time, since the cam 21cc is elastically deformed, a state in which a reaction force f1 is received from the fixed held portion Wa is taken. In the rotational radius direction of the first rotary body 21cp, the reaction force f1 acting on the cam 21cc acts at a position away from the rotational center 21cpc of the first rotary body 21cp, and therefore the first rotary body 21cp rotates clockwise in FIG. 14A. At this time, the first rotary body 21cp is urged in the same direction as the direction in which the first rotary body 21cp is rotated by the internal gear 29 rotating clockwise.

FIG. 15 is a diagram illustrating a state of the internal gear 29 and the coupling portion 21c when the operation portion 400 has rotated in the locking direction R1 from the state of FIG. 14A. FIG. 15 is a diagram illustrating a state of the internal gear 29 and the coupling portion 21c when the operation portion 400 is positioned at the fixation position.

When the operation portion 400 rotates in the locking direction R1 from the state illustrated in FIG. 14A, the first rotary body 21cp further rotates clockwise in the drawing by the reaction force f1 that the cam 21cc provided on the first rotary body 21cp receives from the held portion Wa. Further, as illustrated in FIG. 15, the first rotary body 21cp rotates until an abutting surface Zb1a provided on the tooth Zb1 comes into contact with an abutted surface 21cf (see also FIG. 6B) provided on the coupling base 21cb. That is, the first rotary body 21cp stops in a state in which the abutting surface Zb1a and the abutted surface 21cf are aligned on the same plane.

At this time, the cam 21cc of the first rotary body 21cp is positioned at a pressing position to press the held portion Wa in the −Y direction toward the coupling base 21cb by the pressing surface 21ccb. That is, the cam 21cc is configured to move to the pressing position by the reaction force f1 that the pressing surface 21ccb receives from the held portion Wa and be held at the pressing position after the tooth Za1 of the internal gear 29 is separated from the tooth Zb1 of the gear portion 21cg.

In addition, since the engagement between the held portion Wa and the coupling base 21cb is maintained by the cam 21cc positioned at the pressing position, the coupling portion 21c is in the locked state.

As illustrated in FIG. 14B, in a state in which the held portion Wa is pressed by the cam 21cc positioned at the pressing position, the recess portion Wc of the held portion Wa engages with the protrusion portion 21ci of the coupling base 21cb. As a result of this, the held portion Wa is fixed with respect to the coupling base 21cb.

According to this fixing method, the pressing direction of the held portion Wa by the elastic force of the cam 21cc and the driving direction of the driving wire W (Dc direction, Z direction) are in an orthogonal relationship. Therefore, a force loosening the fixation of the held portion Wa against the elastic force of the cam 21cc is not likely to be generated when driving the driving wire W, and the held portion Wa can be stably fixed with respect to the coupling base 21cb. Further, the held portion Wa can be made less likely to be separated from the coupling portion 21c in the locked state.

To be noted, the tooth Za1 of the internal gear 29 and the tooth Zb2 of the gear portion 21cg are stopped at positions where there is a clearance of the distance Lc therebetween.

When cancelling the fixation between the driving wire W and the coupling portion 21c, the operation portion 400 positioned at the fixed position is rotated in the releasing direction R2. At this time, the internal gear 29 rotates counterclockwise in the drawing from the state illustrated in FIG. 15. When the internal gear 29 rotates counterclockwise, a tooth Za3 of the internal gear 29 abuts a tooth Zb4 of the gear portion 21cg, and the first rotary body 21cp is rotated counterclockwise.

As a result of the internal gear 29 further rotating counterclockwise, the fixation of the driving wire W by the coupling portion 21c is cancelled. At this time, the operation of the internal gear 29 and the first rotary body 21cp is an operation reversed from the operation described above. That is, the fixation of the driving wire W by the coupling portion 21c is cancelled by the operation reversed from the operation for fixing the driving wire W by the coupling portion 21c described above.

The operation described above is performed in each of the first to ninth coupling portions (21c11 to 21c33). That is, in the process of movement of the operation portion 400 from the detachment position to the fixation position, the first to ninth coupling portions (21c11 to 21c33) are switched from the released state to the locked state by the movement (rotation) of the operation portion 400. In the process of the movement of the operation portion 400 from the fixation position to the detachment position, the first to ninth coupling portions (21c11 to 21c33) are switched from the locked state to the released state by the movement (rotation) of the operation portion 400.

A state in which the first to ninth driving wires (W11 to W33) are respectively fixed by the first to ninth coupling portions (21c11 to 21c33) will be referred to as a first state. A state in which the fixation of the first to ninth driving wires (W11 to W33) respectively by the first to ninth coupling portions (21c11 to 21c33) is cancelled will be referred to as a second state.

The first state and the second state are switched in an interlocked manner with the movement of the operation portion 400. That is, the first state and the second state are switched in an interlocked manner with movement of the operation portion 400 between the detachment position and the fixation position.

As described above, the internal gear 29 is configured to operate in an interlocked manner with the operation portion 400 via the joint 28 serving as a transmission member. The internal gear 29 and the joint 28 have a function as an interlocking portion that operates in an interlocked manner with the operation portion 400 such that a first state and a second state are switched in an interlocked manner with the movement of the operation portion 400.

Specifically, in a state in which the catheter unit 100 is attached to the base unit 200, the internal gear 29 and the joint 28 move part (cam 21cc) of the first rotary body 21cp with respect to the held portion Wa in an interlocked manner with the movement of the operation portion 400. As a result of the movement of the cam 21cc, the locked state and the released state of the coupling portion 21c can be switched.

To be noted, the internal gear 29 may be configured to be directly moved by the operation portion 400. In this case, the internal gear 29 has a function as an interlocking portion.

### <Movement of Operation Portion>

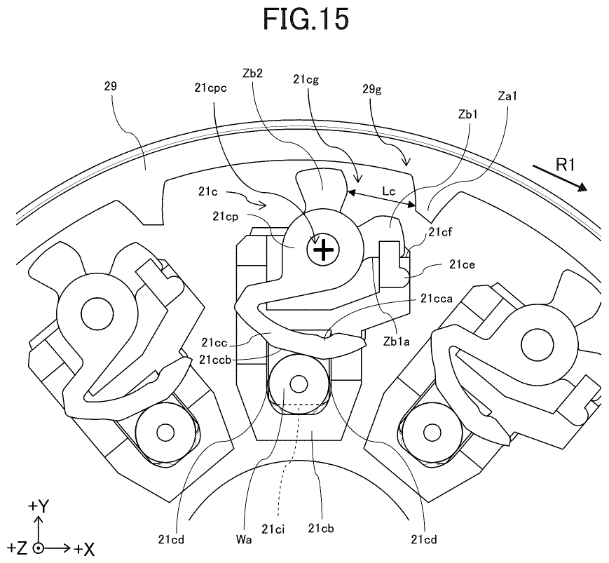

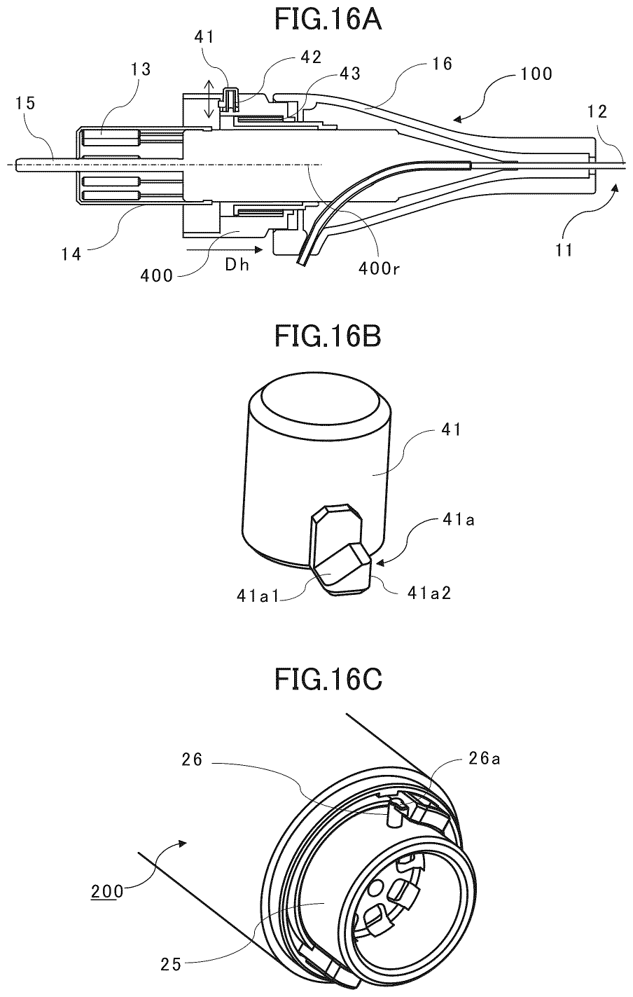

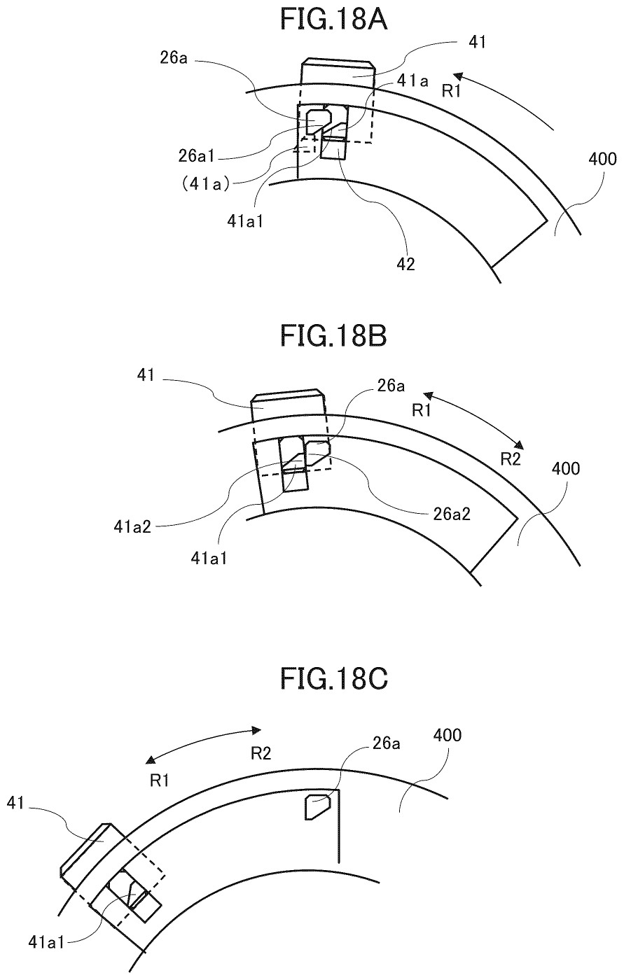

The movement of the operation portion 400 will be described with reference to FIGS. 16A to 16C, 17A to 17C, and 18A to 18C. FIGS. 16A to 16C are explanatory diagrams for describing the operation of the operation portion 400. FIG. 16A is a diagram illustrating a state in which the operation portion 400 is at the detachment position. FIG. 16B is a diagram illustrating a state in which the operation portion 400 is at the release position. FIG. 16C is a diagram illustrating a state in which the operation portion 400 is at the fixation position.

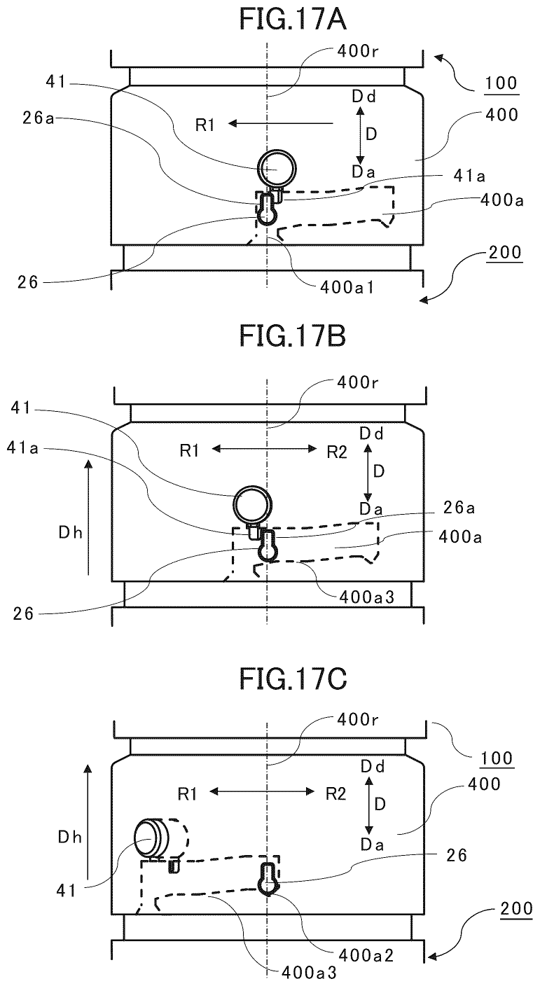

FIGS. 17A to 17C are section views for describing the operation of the operation portion 400. FIG. 17A is a section view illustrating a state in which the operation portion 400 is at the detachment position. FIG. 17B is a section view illustrating a state in which the operation portion 400 is at the release position. FIG. 17C is a section view illustrating a state in which the operation portion 400 is at the fixation position.

As illustrated in FIG. 16A, the catheter unit 100 includes an operation portion urging spring 43 that urges the operation portion 400, a button 41 as a moving member, and a button spring 42 that urges the button 41.

In the present example, the operation portion urging spring 43 is a compression spring. The operation portion 400 is urged in a direction Dh of approaching the near-end cover 16 by the operation portion urging spring 43.

In the present example, the button 41 and the button spring 42 are provided in the operation portion 400. When the operation portion 400 moves to the detachment position, the release position, and the fixation position, the button 41 and the button spring 42 move together with the operation portion 400.

The button 41 is configured to be movable with respect to the operation portion 400 in a direction intersecting with the direction of the rotation shaft 400r of the operation portion 400. The button 41 is urged outward (in a direction away from the rotation shaft 400r) with respect to the catheter unit 100 by the button spring 42.

As will be described later, the movement of the operation portion 400 from the release position to the detachment position is restricted by the button 41. In addition, by moving the button 41 with respect to the operation portion 400, movement of the operation portion 400 from the release position to the detachment position is allowed.

As illustrated in FIG. 16B, the button 41 includes a button protrusion (restricted portion) 41a. The button protrusion 41a includes an inclined surface 41al and a restricted surface 41a2.

As illustrated in FIG. 16C, the base frame 25 of the base unit 200 is provided with the locking shaft 26. The locking shaft 26 includes the locking protrusion (restricting portion) 26a.

In the present example, a plurality of (two in the present example) locking shafts 26 are provided. Each locking shaft 26 may include the locking protrusion 26a, or part of the locking shafts 26 may include the locking protrusion 26a.

Meanwhile, as illustrated in FIGS. 9, 16A, 16B, and 16C, locking grooves 400a that engage with the locking shafts 26 are provided on the inner side of the operation portion 400. The locking grooves 400a extend in a direction different from the attachment/detachment direction D, and extend in the rotational direction of the operation portion 400 in the present example. It can be also said that the locking grooves 400a extend in a direction intersecting with (orthogonal to) the attachment/detachment direction D.

The locking grooves 400a are respectively provided for the plurality of locking shafts 26 in the case where a plurality of locking shafts 26 are provided.

As illustrated in FIG. 17A, when the catheter unit 100 is attached to the base unit 200, a locking shaft 26 engages with a locking groove 400a via an entrance 400al of the locking groove 400a.

At this time, the operation portion 400 is positioned at the detachment position, and the coupling portion 21c is in the released state (see FIGS. 10A and 10B). Therefore, the fixation of the first to ninth driving wires (W11 to W33) respectively by the first to ninth coupling portions (21c11 to 21c33) is cancelled. In addition, as illustrated in FIG. 17A, the button protrusion 41a and the locking protrusion 26a face each other.

When the operation portion 400 is rotated in the locking direction R1 from the detachment position, an inclined surface 41al of the button protrusion 41a abuts an inclined surface 26al of the locking protrusion 26a. The button 41 moves inward (in a direction to approach the rotation shaft 400r) with respect to the operation portion 400 against the urging force of the button spring 42. Then, the button protrusion 41a climbs over the locking protrusion 26a, and the operation portion 400 moves to the release position (see FIG. 17B).

When the operation portion 400 is positioned at the release position, the coupling portion 21c is in the released state (see FIG. 11). Therefore, the fixation of the first to ninth driving wires (W11 to W33) respectively by the first to ninth coupling portions (21c11 to 21c33) is cancelled.

When the operation portion 400 is rotated in the locking direction R1 from the release position, the operation portion 400 moves to the fixation position. As illustrated in FIG. 17C, in a state in which the operation portion 400 is at the fixation position, a positioning portion 400a2 of the locking groove 400a is positioned at a position corresponding to the locking shaft 26. The operation portion 400 is urged in the direction Dh to approach the near-end cover 16 by the operation portion urging spring 43. As a result, the positioning portion 400a2 engages with the locking shaft 26.

In the process of the operation portion 400 moving from the release position to the fixation position, the held portion Wa of the driving wire W is fixed to the coupling portion 21c as described above.

When the operation portion 400 is positioned at the fixation position, the coupling portion 21c is in the locked state (see FIG. 15). Therefore, the first to ninth driving wires (W11 to W33) are respectively fixed to the first to ninth coupling portions (21c11 to 21c33). In this state, the driving force from the wire driving portion 300 can be transmitted to the bending driving portion 13. That is, the driving force respectively from the first to ninth drive sources (M11 to M33) can be respectively transmitted to the first to ninth driving wires (W11 to W33) via the first to ninth coupling portions (21c11 to 21c33).

When the operation portion 400 is at the release position (FIG. 17B), a wall 400a3 defining the locking groove 400a is positioned upstream of the locking shaft 26 in the detachment direction Dd of the catheter unit 100. When the operation portion 400 is at the fixation position (FIG. 17C), the positioning portion 400a2 is positioned upstream of the locking shaft 26 in the detachment direction Dd. As a result, when the operation portion 400 is at the release position or the fixation position, detachment of the catheter unit 100 from the base unit 200 is restricted. In contrast, when the operation portion 400 is at the detachment position (FIG. 17A), the entrance 400al of the locking groove 400a is positioned upstream of the locking shaft 26 in the detachment direction Dd. As a result, detachment of the catheter unit 100 from the base unit 200 is allowed.

When the operation portion 400 is rotated in a releasing direction R2 from the fixation position, the held portion Wa of the driving wire W is released from the coupling portion 21c as described above.

In a state in which the operation portion 400 is positioned at the release position, the restricted surface 41a2 of the button protrusion 41a abuts a restricting surface 26a2 of the locking protrusion 26a (see FIG. 17B). In this state, rotation of the operation portion 400 in the releasing direction R2 is restricted. In addition, detachment of the catheter unit 100 from the base unit 200 is restricted.

As a result of the user pushing the button 41 inward with respect to the operation portion 400 in a state in which the operation portion 400 is positioned at the release position, the restricted surface 41a2 is separated from the restricting surface 26a2, and the button protrusion 41a climbs over the locking protrusion 26a. As a result, rotation of the operation portion 400 in the releasing direction R2 is allowed, and thus the operation portion 400 can move from the release position to the detachment position.

To be noted, in the present example, the number of each of the locking protrusion 26a and the button 41 is one. The medical apparatus 1 may include a plurality of the locking protrusions 26a and the buttons 41.

### <Sequence Control of Catheter Unit Attachment>

As described above, in the present example, after the catheter unit 100 is attached to the base unit 200, the coupling portion 21c can be switched from the released state to the locked state by rotationally operating the operation portion 400.

In design, in the released state illustrated in FIG. 10B, a recess portion center Wd of the held portion Wa and a protrusion portion center 21cj of the coupling base 21cb coincide with each other. That is, after attaching the catheter unit 100 to the base unit 200, an expected position (named position in design) in the Z direction of the held portion Wa in a state before the operation portion 400 is rotationally operated is a position engageable with the coupling base 21cb. Here, the recess portion center Wd is a center position of the recess portion Wc in the Z direction, and the protrusion portion center 21cj is a center position of the protrusion portion 21ci in the Z direction. In addition, the center position of the recess portion Wc is a middle point in the Z direction between the center (surface center) of the first inclined surface Wc1 and the center (surface center) of the second inclined surface Wc2. The center position of the protrusion portion 21ci is a middle point in the Z direction between the center (surface center) of the first inclined surface 21cj1 and the center (surface center) of the second inclined surface 21cj2.

However, the recess portion center Wd of the held portion Wa and the protrusion portion center 21cj of the coupling base 21cb do not necessarily coincide with each other due to variations in production and individual differences of the catheter unit 100 or the base unit 200, that is, due to dimensional tolerance of parts, assembly tolerance, and the like.

A case where the recess portion center Wd of the held portion Wa and the protrusion portion center 21cj of the coupling base 21cb are displaced from each other beyond an allowable range at the time when the catheter unit 100 is attached to the base unit 200 will be considered. In this case, there is a possibility that the recess portion Wc of the held portion Wa does not smoothly engage with the protrusion portion 21ci of the coupling base 21cb even if the operation portion 400 is rotated in the locking direction R1. In this case, for example, there is a possibility that a state (engagement failure) in which the engagement between the recess portion Wc of the held portion Wa and the protrusion portion 21ci of the coupling base 21cb is not established is taken even though the operation portion 400 has been moved to the fixation position, and the driving force of the drive source M is not transmitted to the catheter 46. In addition, for example, if the operation portion 400 is forcibly moved to the fixation position, there is a possibility that a strong load is applied to and damage the cam 21cc and the other parts. Employing a configuration in which the operation of the drive source M is stopped by detecting the engagement failure of the held portion Wa and the coupling base 21cb (fixation failure of the driving wire W) or damage to the apparatus can be also considered, but this requires at least replacement of the catheter unit 100.

Therefore, it is desired that, in the process of attachment of the catheter unit 100 to the base unit 200, the coupling base 21cb is moved (position is adjusted) to a position in the Z direction engageable with the held portion Wa in accordance with the position of the held portion Wa. The “position in the Z direction engageable with the held portion Wa” of the coupling base 21cb is a position where, in a direction (Y direction) intersecting with the Z direction, the first inclined surface 21ci1 (first abutting portion) of the coupling base 21cb opposes the first inclined surface Wc1 (first abutted portion) of the held portion Wa, and the second inclined surface 21ci2 (second abutting portion) of the coupling base 21cb opposes the second inclined surface Wc2 (second abutted portion) of the held portion Wa. By adjusting the position of the coupling base 21cb by aiming to such a position, the held portion Wa and the coupling base 21cb can be more reliably engaged with each other in the case of moving the held portion Wa or the coupling base 21cb in the direction (Y direction) intersecting with the Z direction. To be noted, the target position of the position adjustment may be offset from an ideal engagement position (position where the protrusion portion center 21cj coincides with the recess portion center Wd) of the coupling base 21cb with respect to the held portion Wa within a range not hindering the engagement of the held portion Wa and the coupling base 21cb.

In the present example, after the catheter unit 100 is attached to the base unit 200, before the user moves the operation portion 400 to the fixation position, the recess portion center Wd of the held portion Wa is caused to coincide with the protrusion portion center 21cj of the coupling base 21cb in the position in the Z direction. In the description below, the configuration and operation for position adjustment of the coupling base 21cb in the present example will be described in detail.

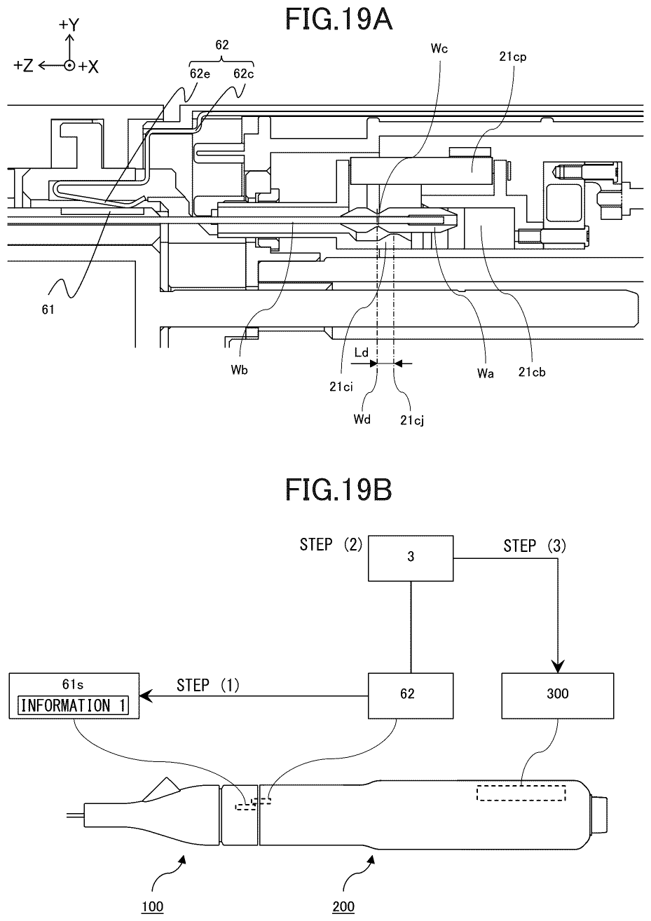

FIG. 19A is a section view illustrating a state of the held portion Wa and the coupling base 21cb in the detachable state of the catheter unit 100. FIG. 19B is a schematic diagram illustrating a configuration related to the sequence control of the position adjustment of the coupling base 21cb in the present example.

As illustrated in FIG. 19A, the catheter unit 100 includes at least one memory device 61 (memory chip) that stores the position information (information 1 in the drawing) of the held portion Wa. The position information of the held portion Wa is data with which the positions in the Z direction of all the held portions Wa measured at least after the unit is assembled can be specified. The position information of the held portion Wa is, for example, a set of numerical values indicating the positional deviation amount of the recess portion center Wd measured for each of the actual held portions Wa with respect to the named position of the recess portion center Wd of the held portion Wa.

Meanwhile, the base unit 200 includes at least one reading portion 62 as a reader for reading the information stored in the memory device 61. For example, the reading portion 62 is electrically connected to the control portion 3 via a cable 5. The reading portion 62 is fixed to the frame (base frame 25) of the base unit 200. To be noted, the reading portion 62 of the present example is a connecting portion (conduction portion) that electrically interconnects the control portion 3 and the memory device 61. Not only the reading portion 62 serving as a connecting portion (conduction portion) but also a reading portion that transmits a command to the memory device 61, processes a signal from the memory device 61, and extracts and transmits necessary information to the control portion 3 may be provided.

To be noted, the catheter unit 100 is preferably stored in a state in which the influence of external force and environment is blocked as much as possible, such that after the measurement of the position of the held portion Wa, the positional relationship is maintained until the catheter unit 100 is attached to the base unit 200.

In addition, the position information of all the held portions Wa can be stored in one memory device 61. In this case, it suffices as long as the base unit 200 includes one reading portion 62 corresponding to the memory device 61.

The reading portion 62 includes an elastically deformable terminal portion 62e (contact portion) to contact a contact portion of the memory device 61, and a conductive line portion 62c through which a signal is communicated between the terminal portion 62e and the control portion 3. The terminal portion 62e is disposed to come into contact with the contact portion of the memory device 61 in a state in which the catheter unit 100 is attached to the base unit 200. In addition, the terminal portion 62e has a margin in the length in the Z direction such that electrical connection with the contact portion of the memory device 61 is guaranteed within the range of the expected maximum positional deviation amount of the held portion Wa.

FIG. 19A illustrates a state in which the recess portion center Wd of the held portion Wa and the protrusion portion center 21cj of the coupling base 21cb do not coincide with each other in the Z direction in the detachable state of the catheter unit 100 (state immediately after attachment to the base unit 200). Specifically, the recess portion center Wd of the held portion Wa is at a position deviated by Ld from the protrusion portion center 21cj of the coupling base 21cb in the +Z direction.

If the operation portion 400 is rotationally operated toward the locking position by a user in the state of FIG. 19A, there is a possibility that an engagement failure of the held portion Wa and the coupling base 21cb occurs as described above. In contrast, in the present example, after the attachment of the catheter unit 100 to the base unit 200 and before the operation portion 400 is rotationally operated toward the locking position, the position adjustment of the coupling base 21cb is performed on the basis of the information read by the control portion 3 from the memory device 61.

The sequence control performed in the present example will be described with reference to FIG. 19B.
Step (1): After electrical connection with the memory device 61 is established, the control portion 3 immediately reads positional information of each held portion Wa from the memory device 61 via the reading portion 62.
Step (2): Next, the control portion 3 calculates movement amounts for moving the coupling bases 21cb respectively corresponding to the held portions Wa on the basis of the read position information. The calculated movement amounts are movement distances for moving the coupling base 21cb for cancelling the positional deviation (Ld of FIG. 19A) in the Z direction between the recess portion center Wd of the held portion Wa and the protrusion portion center 21cj of the coupling base 21cb.
Step (3): Subsequently, the control portion 3 instructs the wire driving portion 300 such that the drive sources M each rotate in a rotational direction and by a rotational amount corresponding to the movement amount calculated in step (2).

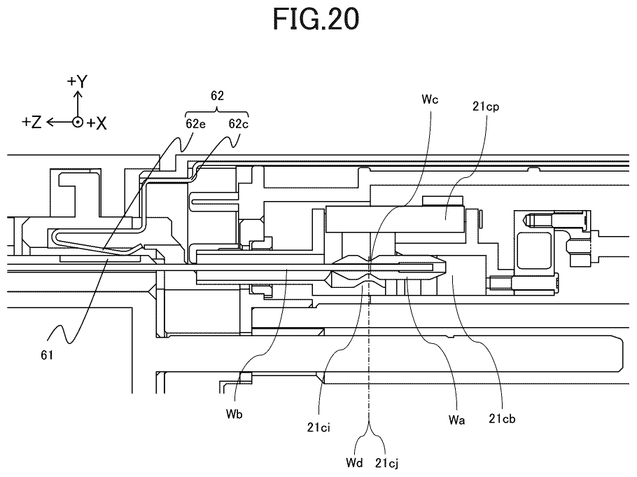

By the sequence control described above, as illustrated in FIG. 20, the coupling base 21cb is driven in the +Z direction so as to cancel the positional deviation (Ld of FIG. 19A) of the held portion Wa. Further, a state in which the recess portion center Wd of the held portion Wa and the protrusion portion center 21cj of the coupling base 21cb approximately coincide with each other (or a state in which at least the positional deviation is reduced to less than Ld) is taken. To be noted, the processing speed of the control portion 3 and the response speed of the drive source M is normally sufficiently faster than the operation of the user, and therefore the position adjustment of the coupling base 21cb by the sequence control is completed before the user starts the rotational operation of the operation portion 400.

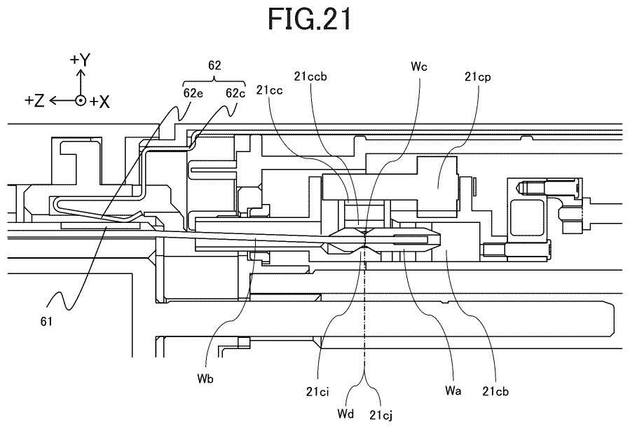

Then, after the position adjustment of the coupling base 21cb, when the user rotationally operates the operation portion 400 toward the locking position, as illustrated in FIG. 21, the cam 21cc moves from the retracted position to the pressing position in an interlocked manner with the rotation of the operation portion 400. Then, the held portion Wa is pressed in the −Y direction by the cam 21cc, the recess portion Wc of the held portion Wa and the protrusion portion 21ci of the coupling base 21cb are engaged with each other, and the driving wire W is locked to the coupling portion 21c.

At this time, since the position of the coupling base 21cb is adjusted by the sequence control described above, the possibility of occurrence of the engagement failure of the held portion Wa and the coupling base 21cb is reduced, and thus the held portion Wa and the coupling base 21cb can be more reliably engaged with each other. That is, according to the present example, the attachment of the bendable unit (catheter unit 100) can be performed more reliably.

In addition, since the operation of the operation portion 400 is performed in a state in which the recess portion center Wd of the held portion Wa and the protrusion portion center 21cj of the coupling base 21cb approximately coincide with each other, the recess portion Wc and the protrusion portion 21ci engage with each other more smoothly. Therefore, the user can stabilize the operational force (operational load) required when the user rotationally operates the operation portion 400, and thus the operability can be improved.

### Modification Example 1

In Example 1 described above, although a configuration in which the electric connection between the control portion 3 and the memory device 61 is established in the process of attachment of the catheter unit 100 has been described, the configuration for the control portion 3 to obtain the position information of the held portion Wa of the catheter unit 100 is not limited to this.

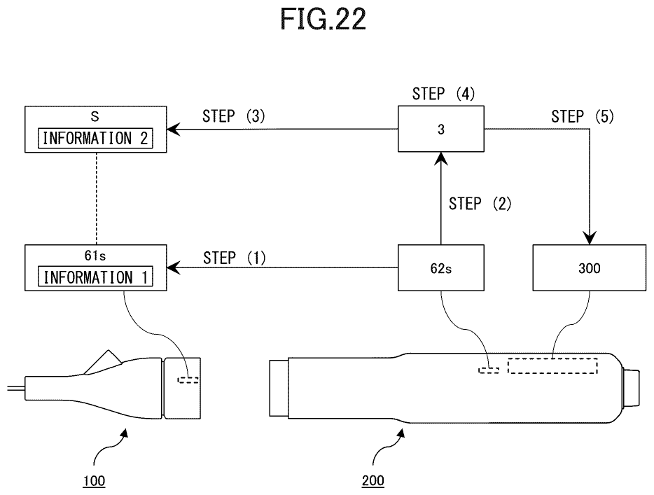

FIG. 22 is a schematic diagram illustrating, as a modification example, a configuration in which information is read from the memory device (storage portion) by contactless communication and the position information of the held portion Wa is obtained from a storage device (server) different from the memory device of the catheter unit 100.

In the present modification example, the catheter unit 100 includes a memory device 61s such as a passive RFID. As identification information for identifying the individual catheter unit 100, information of ID number (information 1 in the drawing) in production is registered in the memory device 61s. That is, unlike in Example 1, numerical values indicating the position information of the held portion Wa and the like are not stored in the memory device 61s. Instead, the ID number of the catheter unit 100, and the position information of each of the held portions Wa of the unit (information 2 in the drawing) are stored in a server S serving as an external information processing apparatus that the control portion 3 is capable of communicating with. The server S holds, in association with the ID number of the unit, the position information of each of the held portions Wa obtained by measurement after the catheter unit 100 is manufactured. The base unit 200 includes a reading portion 62s capable of reading the information of the memory device 61s by contactless communication using an electric wave. The reading portion 62s that performs contactless communication with the memory device 61s is another example of the reader.

Sequence control in the present modification example will be described.
Step (1): When the catheter unit 100 is attached to the base unit 200, the reading portion 62s detects the memory device 61s of the approaching catheter unit and reads the ID number.
Step (2): The reading portion 62s transmits the read ID number to the control portion 3.
Step (3): The control portion 3 having received the ID number from the reading portion 62s accesses the server S, compares the ID number, and thus obtains the position information of the held portion Wa of the catheter unit 100 attached this time.
Step (4): Next, the control portion calculates the movement amounts for moving the coupling bases 21cb respectively corresponding to the held portions Wa on the basis of the position information obtained from the server S.
Step (5): Subsequently, the control portion 3 instructs the wire driving portion 300 such that the drive sources M each rotate in a rotational direction and by a rotational amount corresponding to the movement amount calculated in step (4).

Also according to such a configuration, the attachment of the bendable unit (catheter unit 100) can be performed more reliably.

To be noted, although description has been given assuming that the position information of the held portion Wa is not stored in the memory device 61s in the modification example described above, the position information of the held portion Wa may be stored in the memory device 61s, and the position information of the held portion Wa may be read by the reading portion 62s by contactless communication. In this case, the control portion 3 may calculate the movement amount of the coupling base 21cb on the basis of the position information of the held portion Wa obtained via the reading portion 62s and instruct the wire driving portion 300 similarly to Example 1.

In addition, in Example 1, a configuration in which only the ID number of the catheter unit 100 is stored in the memory device 61 and the position information of the held portion Wa is obtained from the server S on the basis of the ID number read from the memory device 61 by the control portion 3 may be employed.

In addition, the memory devices 61 and 61s described above are examples of a recording medium in which the position information of the held portion Wa or the information required for obtaining the position information is recorded, and a different medium may be used instead of the memory device (semiconductor element). For example, the position information of the held portion Wa included in the catheter unit 100 or the ID number of the catheter unit 100 may be embedded in an image such as a two-dimensional barcode printed on the surface of the catheter unit 100. In this case, as the reading portion, an optical sensor (barcode reader) capable of reading an image can be used.

### Modification Example 2

In the configuration of Example 1, a connecting portion (engagement portion) constituted by the coupling base 21cb and the held portion Wa can be imparted with a breakaway mechanism or a separation mechanism that disconnects the connection between the drive source M and the driving wire W in the case where an overload acts on the driving wire W. It is assumed that, in the engaged state illustrated in FIG. 14B, a load (overload) exceeding a predetermined threshold value acts on the driving wire W by abnormal operation of the drive source M, an external force to the catheter 11, and the like. At this time, the held portion Wa moves in the +Z direction or the −Z direction so as to climb over the protrusion portion 21ci while elastically deforming the cam 21cc, and thus the engagement between the coupling base 21cb and the held portion Wa is cancelled.

As a result of this, the connection between the drive source M and the driving wire W can be disconnected. A possibility that the bending portion 12 of the catheter 11 is bent by an excessively strong force by the abnormality of the drive source M, or the coupling portion 21c or the driving wire W is damaged when the catheter 11 receives a strong external force can be lowered.

In the configuration described above, a force in the Z direction required for moving the held portion Wa such that the engagement between the protrusion portion 21ci and the recess portion Wc is cancelled against the elastic force of the cam 21cc corresponds to the threshold value of the load at which the breakaway mechanism operates. This threshold value is a value larger than an expected maximum value of the tensile force and the compressive force acting on the driving wire W in the bending control of the catheter unit 100 in a normal use state.

To be noted, a configuration in which the coupling base 21cb and the held portion Wa are not imparted with the function of the breakaway mechanism and the drive source M is driven to relieve the load in the case where a load detected by a force detection portion such as a force measurement portion 39 that will be described in Example 2 below exceeds the threshold value may be employed. In addition, the coupling base 21cb and the held portion Wa having the function of the breakaway mechanism may be used in combination with a load relieving function (backdrive control) using a force detection portion. In this case, the threshold value of the load at which the backdrive control operates is set to be lower than the threshold value of the load at which the breakaway mechanism operates.

### Example 2

Next, a second embodiment (Example 2) of the present disclosure will be described. In the description below, illustration of elements having substantially the same configuration and effects as in Example 1 is omitted, or described by giving the same reference signs.

In Example 1, the position adjustment of the coupling base 21cb is performed on the basis of the position information of the held portion Wa included in the catheter unit 100. In the present example, the position adjustment of the coupling base 21cb is performed on the basis of the detection result of the force detection portion that detects the tensile force or compressive force acting on the coupling base 21cb.

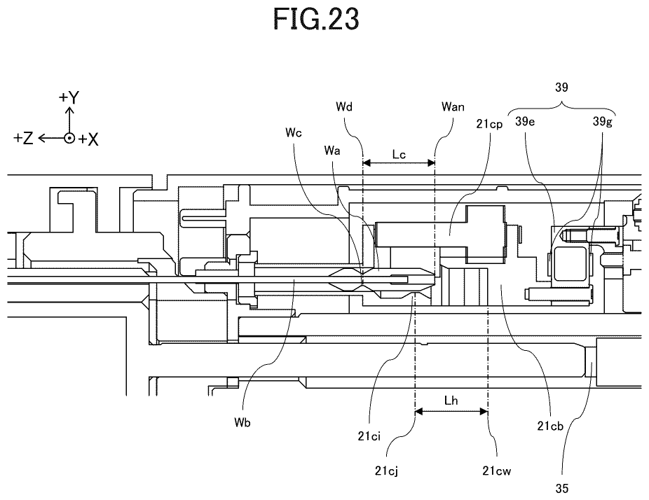

FIG. 23 is a section view illustrating a state (detachable state) immediately after the catheter unit 100 is attached to the base unit 200 in the medical apparatus 1 according to the present example. As a configuration different from Example 1, the coupling base 21cb includes an abutment surface 21cw serving as an abutment portion (contact portion) that the near end of the driving wire W, that is, a near end Wan of the held portion Wa is capable of coming into contact with. The abutment surface 21cw is disposed such that the distance Lh in the Z direction from the protrusion portion center 21cj of the coupling base 21cb to the abutment surface 21cw matches the distance Lc in the Z direction from the recess portion center Wd of the held portion Wa to the near end Wan.

In addition, the wire driving portion 300 of the base unit 200 includes a force measurement portion 39 as a force detection portion (load measurement portion) for monitoring (detecting) the tensile force or the compressive force generated in the driving wire W. The force measurement portion 39 includes a strain element 39e and a strain gauge 39g. The strain element 39e is slightly elastically deformed in accordance with the magnitude of the tensile force or the compressive force in the case where a tensile force or a compressive force is generated in the driving wire W. The force measurement portion 39 detects the elastic deformation of the strain element 39e by the strain gauge 39g, and transmits an electric signal to the control portion 3. As a result of this, the control portion 3 can grasp the tensile force or the compressive force generated in each of the plurality of driving wires W.

To be noted, the force measurement portion 39 can be used as a trigger for control of driving the drive source M in a direction to relieve the load in the case where a load equal to or greater than a predetermined value acts on the driving wire W during use of the medical apparatus 1, that is, during an operation involving movement of the medical apparatus 1 or driving of the bending driving portion 13. This control is referred to as backdrive control in the present example. In the backdrive control, for example, in the case where a large tensile force acts on one of the driving wires W as a result of a far end of the catheter 11 coming into contact with an obstacle or the like, the tensile force is relieved by moving the corresponding coupling base 21cb in the +Z direction. According to this configuration, the possibility that the catheter 11 comes into strong contact with the human body of a patient serving as an insertion target or the driving elements of the driving wire W are damaged can be lowered.

Further, the base unit 200 includes an insertion/removal sensor 35 serving as an attachment detection portion (insertion/removal detection portion) that detects insertion of the catheter unit 100 to a predetermined attachment position in the −Z direction. The predetermined attachment position is a position to which the catheter unit 100 is inserted in the −Z direction with respect to the base unit 200 until it becomes possible to rotationally operate the operation portion 400 from the detachment position to the fixation position.

For the insertion/removal sensor 35, a switch (limit switch) that detects the contact of part of the catheter unit 100 (for example, an end surface of the key shaft 15 in the −Z direction). In addition, as the insertion/removal sensor 35, an optical sensor which includes a light emitting portion and a light receiving portion and in which light is blocked by part of the catheter unit 100 in the case where the catheter unit 100 is positioned at a predetermined attachment position may be used. This is not limiting, and a configuration capable of detecting the position of the catheter unit 100 in the Z direction with respect to the base unit 200 can be used as the insertion/removal detection portion.

In the present example, in a state in which the catheter unit 100 is not attached to the base unit 200, that is, in an attachment standby state, all the coupling bases 21cb are retracted to limit positions on the near-end side (−Z direction side) in the movable ranges. FIG. 23 illustrates the position of the coupling base 21cb in the attachment standby state. The position to which the coupling base 21cb is retracted is a position where the near end Wan of the held portion Wa does not come into contact with the abutment surface 21cw of the coupling base 21cb even in the case where the position of the held portion Wa varies due to the individual difference of the catheter unit 100.

When the insertion/removal sensor 35 detects that the catheter unit 100 has been inserted to a predetermined attachment position, the control portion 3 sends a command to the wire driving portion 300 to start moving all the coupling bases 21cb to the far-end side (+Z direction side). Then, the coupling base 21cb moves to a position where the near end Wan of the held portion Wa comes into contact with the abutment surface 21cw. That is, the control portion 3 of the present example moves the coupling base 21cb (first member) to one side (+Z direction side) in the longitudinal direction such that the abutment surface 21cw (abutment portion) abuts the held portion Wa (second member).

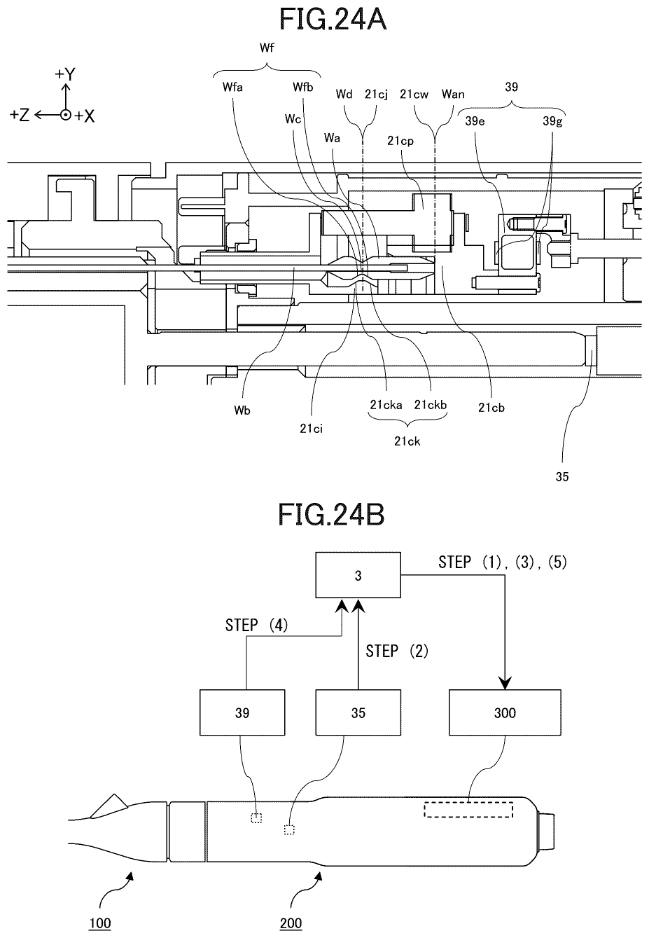

FIG. 24A illustrates a state in which the coupling base 21cb has moved to a position where the near end Wan of the held portion Wa comes into contact with the abutment surface 21cw. If it is attempted to further move the coupling base 21cb to the far-end side (+Z direction side) from this state, since the far-end side of the driving wire W is fixed to a guide ring as described with reference to FIGS. 3A and 3B, a compressive force is generated in the driving wire W, and the strain element 39e starts deforming.

Therefore, after the movement of the coupling base 21cb to the far-end side (+Z direction side) is started, in the case where the compressive force on the driving wire W measured by the force measurement portion 39 has become a first predetermined value or more, movement of the coupling base 21cb corresponding to that driving wire W may be stopped. As a result of this, the coupling base 21cb stops in the vicinity of the position at the time when the near end Wan of the held portion Wa has come into contact with the abutment surface 21cw.

To be noted, the “first predetermined value” described above is a threshold value set in advance such that the presence or absence of the contact between the abutment surface 21cw of the coupling base 21cb and the near end Wan of the held portion Wa can be determined. The first predetermined value can be determined on the basis of the measurement result of the force measurement portion 39 in the case where the coupling base 21cb is repeatedly moved to come into contact with and out of contact from the near end Wan of the held portion Wa. The determined first predetermined value is stored in a storage region in the control portion 3, and is referred to when the control portion 3 executes the sequence control.

As described above, the distance Lh in the Z direction from the protrusion portion center 21cj of the coupling base 21cb to the abutment surface 21cw is configured to match the distance Lc in the Z direction from the recess portion center Wd of the held portion Wa to the near end Wan. Therefore, when the movement of the coupling base 21cb is stopped, the recess portion center Wd of the held portion Wa and the protrusion portion center 21cj of the coupling base 21cb approximately coincide in the Z direction as illustrated in FIG. 24A.

As illustrated in FIG. 24B, the sequence control in the present example is as follows.
Step (1): In a period in which the catheter unit 100 is not attached to the base unit 200, the control portion 3 retracts the coupling base 21cb to a retracted position on the near-end side (−Z direction side) in advance.
Step (2): The insertion/removal sensor 35 detects attachment of the catheter unit 100 to the base unit 200.
Step (3): The control portion 3 instructs the wire driving portion 300 to start moving the coupling base 21cb to the far-end side (+Z direction side) on the basis of the detection by the insertion/removal sensor 35.
Step (4): The control portion 3 determines, on the basis of the detection result of the force measurement portion 39, that a load of a predetermined value (first predetermined value) or more has acted on the driving wire W.
Step (5): Then, the control portion 3 instructs the wire driving portion 300 to stop the movement of the coupling base 21cb.

By the sequence control described above, a state in which the recess portion center Wd of the held portion Wa and the protrusion portion center 21cj of the coupling base 21cb approximately coincide with each other is realized as illustrated in FIG. 24A even if there is positional deviation of the held portion Wa. In addition, since the sequence control is independently executed for each of the plurality of coupling bases 21cb, for each of the held portions Wa, the position of the coupling base 21cb corresponding thereto can be adjusted regardless of the positional difference between the plurality of held portions Wa. To be noted, the processing speed of the control portion 3, the response speed of the drive source M, and the like are normally sufficiently faster than the operation of the user, and therefore the position adjustment of the coupling base 21cb by the sequence control is completed before the user starts the rotational operation of the operation portion 400.

After the position adjustment of the coupling base 21cb, when the user rotationally operates the operation portion 400 toward the locking position, the cam 21cc moves from the retracted position to the pressing position as described above in an interlocked manner with the rotation of the operation portion 400 as described above. Then, the held portion Wa is pressed in the −Y direction by the cam 21cc, thus the recess portion Wc of the held portion Wa and the protrusion portion 21ci of the coupling base 21cb engage with each other, and the driving wire W is locked to the coupling portion 21c.

At this time, since the position of the coupling base 21cb is adjusted by the sequence control described above, the possibility that an engagement failure between the held portion Wa and the coupling base 21cb occurs is lowered, and the held portion Wa and the coupling base 21cb can be caused to more reliably engage with each other. That is, also according to the present example, the attachment of the bendable unit (catheter unit 100) can be performed more reliably.

In addition, since the operation portion 400 is operated in a state in which the recess portion center Wd of the held portion Wa and the protrusion portion center 21cj of the coupling base 21cb approximately coincide with each other, the recess portion Wc and the protrusion portion 21ci engage with each other more smoothly. Therefore, the operational force (operational load) required when the user rotationally operates the operation portion 400 can be stabilized, and the operability can be improved.

To be noted, although the distance Lh in the Z direction from the protrusion portion center 21cj of the coupling base 21cb to the abutment surface 21cw is configured to match the distance Lc in the Z direction from the recess portion center Wd of the held portion Wa to the near end Wan in the present example, this is not limiting. In consideration of the part tolerance and the assembly tolerance, it is preferable that the distance Lh is slightly larger than the distance Lc.

This is because in the case where the actual distance Lh is smaller than the distance Lc due to the part tolerance or the assembly tolerance, there is a possibility that the abutment surface 21cw and the near end Wan interfere with each other when the coupling base 21cb and the held portion Wa are caused to engage with each other. That is, since the abutment surface 21cw and the near end Wan are in a positional relationship to interfere when trying to match the protrusion portion center 21cj and the recess portion center Wd correctly, there is a possibility that the protrusion portion 21ci does not sufficiently fit in the recess portion Wc even when the cam 21cc moves from the retracted position to the pressing position. In addition, there is a possibility that an undesired tensile force acts on the driving wire W in the engaged state.

Regarding the protrusion portion 21ci and the recess portion Wc described above, in the case where the held portion Wa is pressed in the −Y direction by the cam 21cc, a force to relatively move such that the protrusion portion center 21cj and the recess portion center Wd coincide with each other acts between the coupling base 21cb and the held portion Wa. That is, in the case where the recess portion center Wd is displaced to the −Z direction side with respect to the protrusion portion center 21cj, a force to move the held portion Wa to the +Z direction side with respect to the coupling base 21cb is generated by the contact between the first inclined surfaces 21ci1 and Wc1 of the protrusion portion 21ci and the recess portion Wc. Conversely, in the case where the recess portion center Wd is displaced to the +Z direction side with respect to the protrusion portion center 21cj, a force to move the held portion Wa to the −Z direction side with respect to the coupling base 21cb is generated by the contact between the second inclined surfaces 21ci2 and Wc2 of the protrusion portion 21ci and the recess portion Wc.

Therefore, by employing a configuration in which the distance Lh is slightly larger than the distance Lc as described above, the coupling base 21cb and the held portion Wa can relatively move such that the protrusion portion center 21cj and the recess portion center Wd coincide with each other in the process of the cam 21cc moving from the retracted position to the pressing position. As a result of this, a desirable engaged state in which the recess portion Wc of the held portion Wa and the protrusion portion 21ci of the coupling base 21cb are sufficiently fitted together can be realized.

To be noted, in the case where the distance Lh is larger than the distance Lc, the control portion 3 may, after temporarily stopping the movement of the coupling base 21cb in step (3) of the sequence control of Example 2, move the coupling base 21cb by a predetermined distance in the −Z direction and then stop the coupling base 21cb. The predetermined distance is a length corresponding to a difference (Lh-Lc) between the distance Lh and the distance Lc. As a result of this, the protrusion portion center 21cj of the coupling base 21cb and the recess portion center Wd of the held portion Wa can be caused to approximately coincide with each other.

As a modification example, the coupling base 21cb and the held portion Wa may be provided with a function of the breakaway mechanism. In this case, the distance Lh is made larger than the distance Lc such that a space for the held portion Wa to separate from the protrusion portion 21ci to the −Z direction side is secured. For example, the distance Lh is set such that the far end of the held portion Wa is positioned more on the −Z direction side than the protrusion portion 21ci of the coupling base 21cb in a state in which the near end Wan of the held portion Wa is abutting the abutment surface 21cw of the coupling base 21cb. Also in this case, by the above-described control of moving the coupling base 21cb by a predetermined distance in the −Z direction after temporarily stopping the movement of the coupling base 21cb, the position of the coupling base 21cb can be adjusted.

In addition, as a modification example, a means different from the insertion/removal detection portion may be used as the trigger of start of movement of the coupling base 21cb to the far-end side (+Z direction side). For example, when the attachment of the catheter unit 100 is completed, a similar operation can be realized by inputting a command to start the movement of the coupling base 21cb via a user interface such as the input apparatus 3b.

### Example 3

Next, a third embodiment (Example 3) of the present disclosure will be described. In the description below, illustration of elements having substantially the same configuration and effects as in Example 1 or 2 is omitted, or described by giving the same reference signs.

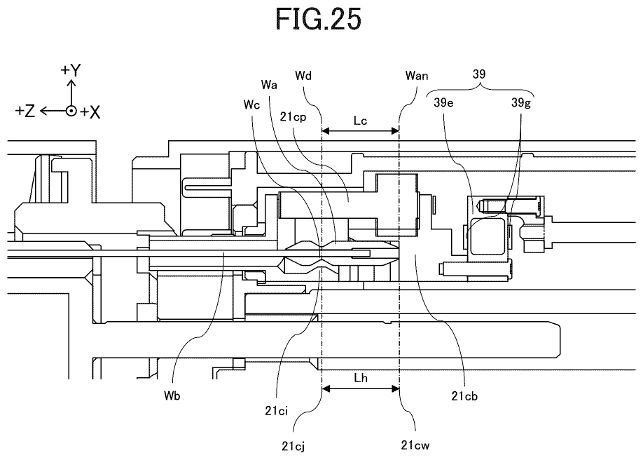

FIG. 25 is a section view illustrating a state in the middle of attachment of the catheter unit 100 to the base unit 200 in Example 3.

Also in Example 3, similarly to Example 2, the coupling base 21cb includes an abutment surface 21cw capable of coming into contact with the near end Wan of the held portion. The abutment surface 21cw is disposed such that the distance Lh in the Z direction from the protrusion portion center 21cj of the coupling base 21cb in the Z direction to the abutment surface 21cw matches the distance Lc in the Z direction from the recess portion center Wd of the held portion Wa to the near end Wan.

In addition, similarly to Example 2, the wire driving portion 300 of the base unit 200 includes the force measurement portion 39 as a force detection portion (load measurement portion) for monitoring (detecting) the tensile force or the compressive force generated in the driving wire W. The force measurement portion 39 is used for the backdrive control described in Example 2 during use of the medical apparatus 1.

In Example 3, this backdrive control is also applied to attachment of the catheter unit 100.

In the present example, in a state in which the catheter unit 100 is not attached to the base unit 200, that is, in the attachment standby state, all the coupling bases 21cb are retracted to limit positions on the far-end side (+Z direction side) in the movable range. FIG. 25 illustrates the position of the coupling base 21cb in the attachment standby state.

When the catheter unit 100 is attached to the base unit 200 in this state, as illustrated in FIG. 25, the near end Wan of the held portion Wa comes into contact with the abutment surface 21cw before the catheter unit 100 reaches the attachment position described above.

If the user further moves the catheter unit 100 in the attachment direction (−Z direction) from the state of FIG. 25, since the far end of the driving wire W is fixed to the guide ring as illustrated with reference to FIGS. 3A and 3B, a compressive force is generated in the driving wire W, and the strain element 39e starts deforming.

Therefore, in the case where the compressive force of the driving wire W measured by the force measurement portion 39 is a second predetermined value or more, the coupling base 21cb corresponding to the driving wire W may be moved to the near-end side (−Z direction side). That is, the position of the coupling base 21cb may be adjusted in accordance with the movement of the held portion Wa at the time of attachment of the catheter unit 100 by control similar to the backdrive control during the use of the medical apparatus 1. That is, the control portion 3 of the present example moves the coupling base 21cb (first member) to the other side (−Z direction) in the longitudinal direction in the case where the abutment surface 21cw (abutment portion) abuts the held portion Wa (second member).

The second predetermined value described above is a threshold value of a load at which the backdrive control operates in the sequence control during attachment of the catheter unit 100. The second predetermined value is set to a value smaller than the threshold value of the load at which the backdrive control operates during the use of the medical apparatus 1. As a result of this, the movement of the coupling base 21cb can be quickly started when the near end Wan of the held portion Wa comes into contact with the abutment surface 21cw during the attachment of the catheter unit 100.

After the movement of the coupling base 21cb to the near-end side (−Z direction side) is started, the movement of the coupling base 21cb is continued until the load measured by the force measurement portion 39 becomes smaller than the second predetermined value. The movement of the coupling base 21cb is stopped when the load becomes smaller than a preset value.

Therefore, if the user tries to attach the catheter unit 100 to the base unit 200, the user feels resistance against the attachment for a moment when the near end Wan of the held portion Wa comes into contact with the abutment surface 21cw. In addition, after such resistance has occurred, occurrence of a large resistance is suppressed by the backdrive control until the catheter unit 100 reaches the attachment position, and the user can perform the attachment operation with a constant operation feel.

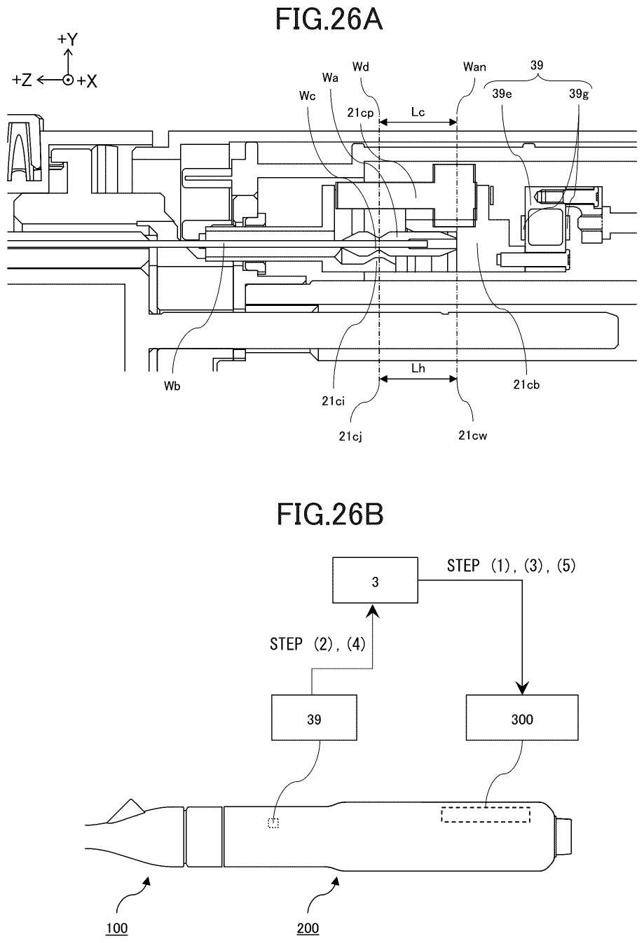

In addition, during the attachment operation of the catheter unit 100, the near end Wan of the held portion Wa and the abutment surface 21cw move together while in contact with each other. When the catheter unit 100 is stopped, the load measured by the force measurement portion 39 becomes smaller than the second predetermined value, and therefore the held portion Wa and the coupling base 21cb stop in a state in which the near end Wan of the held portion Wa and the abutment surface 21cw are in contact with or close to each other. Therefore, when the movement of the coupling base 21cb is stopped, the recess portion center Wd of the held portion Wa and the protrusion portion center 21cj of the coupling base 21cb approximately coincide with each other in the Z direction as illustrated in FIG. 26A.

As illustrated in FIG. 26B, the sequence control in the present example is as follows.
Step (1): In a period in which the catheter unit 100 is not attached to the base unit 200, the control portion 3 retracts the coupling base 21cb to a retracted position on the far-end side (+Z direction side) in advance.
Step (2): In the process of attachment of the catheter unit 100 to the base unit 200, the control portion 3 determines that a load of a predetermined value (second predetermined value) or more has acted on the driving wire W on the basis of the measurement result of the force measurement portion 39.
Step (3): Then, the control portion 3 instructs the wire driving portion 300 to start moving the coupling base 21cb to the near-end side (−Z direction side).
Step (4): The control portion 3 determines, on the basis of the detection result of the force measurement portion 39, that the load on the driving wire W has become smaller than the predetermined value (second predetermined value).
Step (5): Then, the control portion 3 instructs the wire driving portion 300 to stop the movement of the coupling base 21cb.

By the sequence control described above, a state in which the recess portion center Wd of the held portion Wa and the protrusion portion center 21cj of the coupling base 21cb approximately coincide with each other is realized as illustrated in FIG. 26A even if there is positional deviation of the held portion Wa. In addition, since the sequence control is independently executed for each of the plurality of coupling bases 21cb, for each of the held portions Wa, the position of the coupling base 21cb corresponding thereto can be adjusted regardless of the positional difference between the plurality of held portions Wa. To be noted, the processing speed of the control portion 3, the response speed of the drive source M, and the like are normally sufficiently faster than the operation of the user, and therefore the position adjustment of the coupling base 21cb by the sequence control is completed before the user starts the rotational operation of the operation portion 400.

After the position adjustment of the coupling base 21cb, when the user rotationally operates the operation portion 400 toward the locking position, the cam 21cc moves from the retracted position to the pressing position in an interlocked manner with the rotation of the operation portion 400 as described above. Then, the held portion Wa is pressed in the −Y direction by the cam 21cc, thus the recess portion Wc of the held portion Wa and the protrusion portion 21ci of the coupling base 21cb engage with each other, and the driving wire W is locked to the coupling portion 21c.

At this time, since the position of the coupling base 21cb is adjusted by the sequence control described above, the possibility that an engagement failure between the held portion Wa and the coupling base 21cb occurs is reduced, and the held portion Wa and the coupling base 21cb can be caused to more reliably engage with each other. That is, also according to the present example, the attachment of the bendable unit (catheter unit 100) can be performed more reliably.

In addition, according to the configuration of the present example, from the viewpoint of performing the position adjustment of the coupling base 21cb, there is a merit that the insertion/removal detection portion described in Example 2 does not need to be provided.

In addition, in the present example, the position adjustment of the coupling base 21cb is completed approximately simultaneously with the arrival of the catheter unit 100 at the attachment position, and the rotational operation of the operation portion 400 becomes possible. Therefore, in Examples 1 and 2, the possibility that a waiting time before the rotational operation of the operation portion 400 becomes possible occurs after the arrival of the catheter unit 100 at the attachment position depending on the movement amount of the coupling base 21cb in the sequence control can be lowered. As a result of this, the operability for the user can be further improved.

To be noted, also in the present example, it is preferable that the distance Lh is set to be slightly larger than the distance Lc. As a result of this, a desirable engaged state in which the recess portion Wc of the held portion Wa and the protrusion portion 21ci of the coupling base 21cb are sufficiently fitted together can be realized.

To be noted, in the case where the distance Lh is larger than the distance Lc, the control portion 3 may further additionally move the coupling base 21cb by a predetermined distance in the −Z direction from a time point at which it is determined that the load of the driving wire W has become smaller than the predetermined value (second predetermined value) in step (3) of the sequence control of Example 3. The predetermined distance is a length corresponding to the difference (Lh-Lc) between the distance Lh and the distance Lc. The protrusion portion center 21cj of the coupling base 21cb and the recess portion center Wd of the held portion Wa can be caused to approximately coincide with each other.

As a modification example, the coupling base 21cb and the held portion Wa may be provided with a function of the breakaway mechanism. In this case, the distance Lh is made larger than the distance Lc such that a space for the held portion Wa to separate from the protrusion portion 21ci to the −Z direction side. Also in this case, by the above-described control of additionally moving the coupling base 21cb by a predetermined distance in the −Z direction also after the load on the driving wire W has become smaller than the predetermined value (second predetermined value), the position of the coupling base 21cb can be adjusted.

In addition, although the contact position on the held portion Wa side with respect to the abutment surface 21cw of the coupling base 21cb is set to the near end Wan in Examples 2 and 3, this is not limiting. For example, the held portion Wa may be provided with a flange shape, and the coupling base 21b or a different member in the coupling portion 21c may be provided with an abutment surface configured to abut this flange shape. Also in such a configuration, control similar to the sequence control described in Examples 2 and 3 can be realized.

### Other Examples

In Examples 1 to 3, a configuration in which the protrusion portion 21ci and the recess portion Wc are fit together has been described as an example of a configuration in which the driving force of the drive source M is transmitted to the driving wire W by mechanical engagement (physical contact) between the coupling base 21cb (first member) and the held portion Wa (second member). The configuration for engaging the first member and the second member with each other such that drive can be transmitted is not limited to this.

For example, a protrusion portion (for example, an annular protrusion portion) may be provided on the outer periphery of the held portion Wa, and a recess portion that fits with this protrusion portion may be provided on the coupling base 21cb.

In addition, two protrusion portions adjacent to each other in the extending direction (longitudinal direction, Z direction) of the driving wire W may be disposed on one of the held portion Wa and the coupling base 21cb, and a protrusion portion that fits in a recess portion formed between these two may be provided on the other of the held portion Wa and the coupling base 21cb.

In addition, a portion where the held portion Wa receives a force in the +Z direction from the coupling base 21cb and a portion where the held portion Wa receives a force in the −Z direction from the coupling base 21cb may be away from each other. For example, a first protrusion portion and a second protrusion portion may be provided at a distance from each other in the Z direction on the coupling base 21cb. In addition, a third protrusion portion that the first protrusion portion abuts from the +Z direction side and a fourth protrusion portion that the second protrusion portion abuts from the −Z direction side are disposed on the held portion Wa. In this case, the third protrusion portion functions as a first abutted portion capable of receiving a force toward one side (+Z direction side) in the longitudinal direction of the driving wire W by being abutted by the first protrusion portion serving as a first abutting portion. In addition, the fourth protrusion portion functions as a second abutted portion capable of receiving a force toward an other side (−Z direction side) in the longitudinal direction of the driving wire W by being abutted by the second protrusion portion serving as a second abutting portion.

As described above, it suffices as long as a configuration in which, regardless of the specific shapes of the first member and the second member, an engaged state in which the first member and the second member abut each other at at least two positions is achieved, and thus forces to the one side and the other side in the longitudinal direction of the driving wire W are transmitted from the first member to the second member is employed. In such a configuration, by performing the position adjustment of the first member in accordance with the position of the second member of the bendable unit by the sequence control described in each example, an effect similar to each example can be obtained.

In addition, in each example described above, a configuration using a cam mechanism has been described as an example of a configuration in which the first member and the second member are engaged with each other in an interlocked manner with the operation of the operation portion 400 has been described. This is not limiting, and for example, a configuration in which the first member and the second member are engaged with each other by operating the actuator such as a solenoid in accordance with a button operation of the operation portion 400, a command signal from the control portion 3, or the like after the attachment of the catheter unit 100 may be employed.

### OTHER EMBODIMENTS

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

### INDUSTRIAL APPLICABILITY

The present invention can be widely implemented in a medical system including a bendable unit capable of bending such as a catheter unit.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

What is claimed is:

1. A medical system comprising:
a driving unit including a drive source and a first member connected to the drive source;
a bendable unit that includes a bending portion capable of bending, a linear body configured to bend the bending portion, and a second member connected to the linear body and that is attachable to and detachable from the driving unit; and
a control portion configured to control the drive source,
wherein the second member includes a first abutted portion and a second abutted portion,
wherein the first member includes a first abutting portion configured to abut the first abutted portion and press the second member toward one side in a longitudinal direction of the linear body, and a second abutting portion configured to abut the second abutted portion and press the second member toward an other side in the longitudinal direction,
wherein, in a state in which the bendable unit is attached to the driving unit, the first member and the second member are configured to engage with each other such that the first abutting portion and the second abutting portion respectively abut the first abutted portion and the second abutted portion so that the first member and the second member integrally move in the longitudinal direction, and
wherein the control portion is configured to, in a case where the bendable unit is attached to the driving unit, move the first member toward a position in the longitudinal direction where the first member is capable of engaging with the second member, by using the drive source and in accordance with a position of the second member in the longitudinal direction.

2. The medical system according to claim 1,
wherein the bendable unit includes a recording medium in which position information of the second member is recorded,
wherein the medical system further comprises a reading portion configured to read the position information from the recording medium in a case where the bendable unit is attached to the driving unit, and
wherein the control portion is configured to determine a movement amount of the first member on a basis of the position information read by the reading portion.

3. The medical system according to claim 1,
wherein the bendable unit includes a recording medium in which identification information of the bendable unit is recorded,
wherein the medical system further comprises a reading portion configured to read the identification information from the recording medium in a case where the bendable unit is attached to the driving unit, and
wherein the control portion is configured to obtain position information of the first member by communicating with an external information processing apparatus on a basis of the identification information read by the reading portion, and determine a movement amount of the first member on a basis of the obtained position information.

4. The medical system according to claim 2,
wherein the reading portion includes a contact portion configured to come into contact with the recording medium in a case where the bendable unit is attached to the driving unit, and
wherein the recording medium is a memory device configured to be electrically connected to the control portion via the contact portion.

5. The medical system according to claim 2,
wherein the recording medium is a memory device configured such that information thereof is read by the reading portion with contactless communication.

6. The medical system according to claim 1,
wherein the driving unit includes a force detection portion configured to detect a force in the longitudinal direction acting between the first member and the second member, and
wherein the control portion is configured to move the first member on a basis of a detection result of the force detection portion.

7. The medical system according to claim 6,
wherein the first member includes an abutment portion that opposes part of the second member in the longitudinal direction, and
wherein the control portion is configured to,
in a period in which the bendable unit is not attached to the driving unit, position the first member at a position where the abutment portion does not come into contact with the second member even if the bendable unit is attached to the driving unit, and
in a case where the bendable unit is attached to the driving unit, move the first member toward the one side in the longitudinal direction such that the abutment portion abuts the second member, on a basis of the detection result of the force detection portion.

8. The medical system according to claim 7,
wherein the first member is configured to engage with the second member at a position in the longitudinal direction where the abutment portion abuts the second member, and
wherein the control portion is configured to stop movement of the first member in a case where the force detection portion has detected that a compressive force of a predetermined value or more has acted between the first member and the second member after the movement of the first member toward the one side in the longitudinal direction has been started.

9. The medical system according to claim 7,
wherein the first member is configured to engage with the second member at a position where the first member has moved by a predetermined distance, toward the other side in the longitudinal direction with respect to the second member, from a position in the longitudinal direction where the abutment portion abuts the second member, and
wherein the control portion is configured to, after movement of the first member toward the one side in the longitudinal direction is started, temporarily stop the movement of the first member in a case where the force detection portion has detected that a compressive force of a first predetermined value or more has acted between the first member and the second member, and then stop the movement of the first member in a state in which the first member has been moved by the predetermined distance toward the other side in the longitudinal direction.

10. The medical system according to claim 7,
wherein the driving unit includes an attachment detection portion configured to detect that the bendable unit has been attached to a predetermined attachment position, and
wherein the control portion is configured to start movement of the first member toward the one side in the longitudinal direction in a case where the attachment detection portion has detected that the bendable unit has been attached to the attachment position.

11. The medical system according to claim 6,
wherein the first member includes an abutment portion that opposes part of the second member in the longitudinal direction, and
wherein the control portion is configured to,
in a period in which the bendable unit is not attached to the driving unit, position the first member at a position where the abutment portion comes into contact with the second member in a middle of attachment of the bendable unit to the driving unit, and
in a case where the bendable unit is attached to the driving unit, move the first member toward the other side in the longitudinal direction in a case where the abutment portion has abutted the second member, on a basis of the detection result of the force detection portion.

12. The medical system according to claim 11,
wherein the first member is configured to engage with the second member at a position in the longitudinal direction where the abutment portion abuts the second member, and
wherein the control portion is configured to start movement of the first member toward the other side in the longitudinal direction in a case where the force detection portion has detected that a compressive force of a second predetermined value or more has acted between the first member and the second member, and then stop the movement of the first member in a case where the force detection portion has detected that the force acting between the first member and the second member has become smaller than the second predetermined value.

13. The medical system according to claim 11,
wherein the first member is configured to engage with the second member at a position where the first member has moved by a predetermined distance toward the other side in the longitudinal direction with respect to the second member from a position in the longitudinal direction where the abutment portion abuts the second member, and
wherein the control portion is configured to start movement of the first member toward the other side in the longitudinal direction in a case where the force detection portion has detected that a compressive force of a second predetermined value or more has acted between the first member and the second member, and then stop the movement of the first member in a state in which the first member has been further moved by the predetermined distance toward the other side in the longitudinal direction from a time point when the force detection portion has detected that the force acting between the first member and the second member has become smaller than the second predetermined value.

14. The medical system according to claim 6,
wherein the control portion is configured to, in a case of controlling the drive source to bend the bending portion after the bendable unit has been attached to the driving unit and the first member and the second member have been engaged, reduce a load acting on the linear body in a case where the force detection portion has detected that a load of a predetermined value or more has acted on the linear body.

15. The medical system according to claim 1,
wherein the driving unit includes a plurality of drive sources and a plurality of first members respectively connected to the plurality of drive sources,
wherein the bendable unit includes a plurality of linear bodies corresponding to the plurality of drive sources, and a plurality of second members respectively connected to the plurality of linear bodies and configured to respectively engage with the plurality of first members, and
wherein the control portion is configured to, in a case where the bendable unit is attached to the driving unit, independently control the plurality of drive sources in accordance with respective positions of the plurality of second members in the longitudinal direction and thus move each of the plurality of first members to a position in the longitudinal direction where the first member is capable of engaging with a corresponding one of the second members.

16. The medical system according to claim 1,
wherein the bendable unit includes an operation portion configured to be rotationally operated,
wherein the first member and the second member are configured to be engaged with each other as a result of the operation portion being rotationally operated in a state in which the bendable unit is attached to the driving unit.

17. The medical system according to claim 16,
wherein the driving unit includes a cam configured to rotate to a pressing position where the cam presses the second member to engage the second member with the first member and to a retracted position where the cam is retracted from the second member, and an interlocking portion configured to rotate the cam in an interlocked manner with a rotational operation of the operation portion.

18. The medical system according to claim 17,
wherein the operation portion is rotationally operated about an axis extending in the longitudinal direction, and
wherein the cam is configured to press the second member in a direction orthogonal to the longitudinal direction in a case of moving from the retracted position to the pressing position.

19. The medical system according to claim 1,
wherein one of the first member and the second member includes a protrusion portion protruding in a direction intersecting with the longitudinal direction,
wherein an other of the first member and the second member includes a recess portion configured to fit with the protrusion portion,
wherein the first abutting portion and the second abutting portion are part of the protrusion portion or the recess portion provided on the first member, and
wherein the first abutted portion and the second abutted portion are part of the protrusion portion or the recess portion provided on the second member.

20. The medical system according to claim 19,
wherein the first member includes the recess portion formed in an annular shape on an outer peripheral portion of the first member, and
wherein the second member includes the protrusion portion protruding in a direction orthogonal to the longitudinal direction.
